(19) United States
(12) Patent Application Publication
(10) Pub. No.: US 2024/0002932 A1
(43) Pub. Date: Jan. 4, 2024

(54) CLICK CHEMISTRY-BASED DNA PHOTO-LIGATION FOR MANUFACTURING OF HIGH-RESOLUTION DNA ARRAYS

(71) Applicant: 10x Genomics, Inc., Pleasanton, CA (US)

(72) Inventors: Jonathan CHENG, Dublin, CA (US); David Michael Patterson, Oakland, CA (US)

(21) Appl. No.: 18/343,190

(22) Filed: Jun. 28, 2023

Related U.S. Application Data
(60) Provisional application No. 63/356,921, filed on Jun. 29, 2022.

(51) Int. Cl. C12Q 1/6874; C12Q 1/6841

(52) U.S. Cl. CPC C12Q 1/6874; C12Q 2600/16; C12Q 1/6841

(57) ABSTRACT

The present disclosure relates in some aspects to methods and compositions for light-controlled in situ surface patterning of a substrate using a Click chemistry reaction. In some embodiments, a method disclosed herein comprises a copper-catalyzed alkyne-azide (CuAAC) click chemistry reaction for photocontrollable ligation of nucleic acid molecules. A large diversity of barcodes can be created in molecules on the substrate via sequential rounds of light exposure, hybridization, and ligation.

### CROSS-REFERENCE TO RELATED APPLICATIONS

This application claims priority to U.S. Provisional Patent Application No. 63/356,921, filed Jun. 29, 2022, entitled “Click Chemistry-based DNA Photo-ligation for Manufacturing of High-resolution DNA Arrays,” which is herein incorporated by reference in its entirety for all purposes.

### FIELD

The present disclosure relates in some aspects to methods for manufacturing a molecular array using a click reaction and the molecular array generated in situ on a substrate.

### BACKGROUND

Arrays of nucleic acids are an important tool in the biotechnology industry and related fields. These nucleic acid arrays, in which a plurality of distinct or different nucleic acids are positioned on a solid support surface in the form of an array or pattern, find use in a variety of applications, including gene expression analysis, drug screening, nucleic acid sequencing, mutation analysis, and the like.

A feature of many arrays that have been developed is that each of the distinct nucleic acids of the array is stably attached to a discrete location on the array surface, such that its position remains constant and known throughout the use of the array. Stable attachment is achieved in a number of different ways, including covalent bonding of a nucleic acid polymer to the support surface and non-covalent interaction of the nucleic acid polymer with the surface.

There are two main ways of producing nucleic acid arrays in which the immobilized nucleic acids are covalently attached to the substrate surface, i.e., via in situ synthesis in which the nucleic acid polymer is grown on the surface of the substrate in a step-wise, nucleotide-by-nucleotide fashion, or via deposition of a full, presynthesized nucleic acid/polypeptide, cDNA fragment, etc., onto the surface of the array.

While nucleic acid arrays have been manufactured using in situ synthesis techniques, applications in the field of genomics and high throughput screening have fueled the demand for precise chemistry and high fidelity of the synthesized oligonucleotides. Accordingly, there is continued interest in the development of new methods for producing nucleic acid arrays in situ. Provided are methods, uses and articles of manufacture that meet such needs.

### SUMMARY

In some embodiments, provided herein is a method for providing an array, comprising: (a) contacting a lawn of polynucleotides immobilized on a substrate with a first barcode sequence, wherein the polynucleotides comprise a 3′-alkyne or 3′-azido group, and wherein the first barcode sequence comprises an oligonucleotide with a 5′-azido or 5′-alkyne group that is a reaction partner for a Click reaction with the 3′-alkyne or 3′-azido group of the immobilized polynucleotides; (b) masking a first region of the lawn of polynucleotides on the substrate such that only a second region of the lawn of polynucleotides on the substrate are capable of being exposed to light; (c) irradiating the second region with a first ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the second region with the 5′-azido or 5′-alkyne groups of the first barcode sequence to generate a Click product comprising a 1,2,3-triazole group, wherein molecules of the first barcode sequence in proximity to the immobilized polynucleotides in the first region are not catalyzed by the Cu(I) to undergo the Click reaction, thereby providing on the substrate an array comprising the first and second regions of the lawn of polynucleotides, wherein the immobilized polynucleotides in the second region is barcoded with the first barcode sequence and the immobilized polynucleotides in the first region is not barcoded with the first barcode sequence. In some embodiments, diffusion of the Cu(I) generated in the second region is limited such that the Cu(I) does not catalyze the Click reaction between the immobilized polynucleotides in the first region and the first barcode sequence, even though molecules of the first barcode sequence (which can be applied to and have access to the immobilized polynucleotides in the first region and in the second region) can be brought into proximity to the immobilized polynucleotides in the first region, for instance, by hybridization to a splint. The splint, like the molecules of the first barcode sequence, can be applied to and have access to the immobilized polynucleotides in the first region and in the second region.

In some embodiments, provided herein is a method for providing an array, comprising: (a) contacting a lawn of polynucleotides (e.g., oligonucleotides) immobilized on a substrate with a first barcode sequence, wherein the polynucleotides comprise a 3′-alkyne or 3′-azido group, and wherein the first barcode sequence comprises an oligonucleotide with a 5′-azido or 5′-alkyne group that is a reaction partner for a Click reaction with the 3′-alkyne or 3′-azido group of the immobilized polynucleotides; (b) masking a first region of the immobilized polynucleotides on the substrate such that only a second region of the immobilized polynucleotides on the substrate is capable of being exposed to light; (c) irradiating the second region of immobilized polynucleotides on the substrate with a first ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the second region with the 5′-azido or 5′-alkyne groups of the first barcode sequence to generate a Click product comprising a 1,2,3-triazole group, thereby providing on the substrate an array comprising the first and second regions of the immobilized polynucleotides, wherein the second region of immobilized polynucleotides are barcoded with the first barcode sequence and the first region of immobilized polynucleotides are not barcoded with the first barcode sequence.

In any of the embodiments herein, the immobilized polynucleotides can comprise a 3′-alkyne group. In some embodiments, the first barcode sequence comprises a 5′-azido group that reacts with the 3′-alkyne group of the immobilized polynucleotides, thereby forming a Click reaction product. In some such embodiments, the first barcode sequence also comprises a 3′-alkyne group that is available for a Click reaction with a 5′-azido group on other barcodes (e.g., second and third barcodes) in subsequent rounds of building the array.

In any of the embodiments herein, the immobilized polynucleotides can comprise a 3′-azido group. In some such embodiments, the first barcode sequence can comprises a 5′-alkyne group that reacts with the 3′-azido group of the immobilized polynucleotides, thereby forming a Click reaction product. In some such embodiments, the first barcode sequence also comprises a 3′-azido group that is available for a Click reaction with a 5′-alkyne group on other barcodes (e.g., second and third barcodes) in subsequent rounds of building the array.

In any of the embodiments herein, said contacting in step (a) can comprise flowing the first barcode over the lawn of immobilized polynucleotides.

In any of the embodiments herein, the method can further comprise contacting the lawn of immobilized polynucleotides with a splint, wherein the splint can comprise a first nucleotide sequence that is complementary to a nucleotide sequence on the immobilized polynucleotides. In some embodiments, the splint comprises a second nucleotide sequence that is complementary to a nucleotide sequence on the first barcode. In some embodiments, the immobilized polynucleotides can be ligated to the first barcode using the splint as template. In some embodiments, the method further comprises removing the splint after the ligation. In any of the embodiments herein, the method can comprise removing i) the splint and ii) molecules of the first barcode sequence that are brought into proximity to the immobilized polynucleotides in the first region but are not ligated to the immobilized polynucleotides in the first region, after the ligation of molecules of the first barcode sequence to the immobilized polynucleotides in the second region.

In any of the embodiments herein, contacting the splint with the lawn of immobilized polynucleotides can comprise flowing the splint over the lawn of immobilized polynucleotides.

In any of the embodiments herein, the Cu (II) compound can be copper sulfate.

In any of the embodiments herein, the method can further comprise contacting the lawn of polynucleotides with a free radical scavenger. In some embodiments, the Cu (II) compound and the photoinitiator can be flowed over the immobilized lawn of polynucleotides in a solution. In some embodiments, the solution comprises DMSO.

In any of the embodiments herein, all of the polynucleotides immobilized on substrate can be identical in sequence.

In any of the embodiments herein, the polynucleotides immobilized on the substrate prior to the irradiating step can be between about 4 and about 50 nucleotides in length.

In any of the embodiments herein, the immobilized polynucleotides can be double stranded. In any of the embodiments herein, the immobilized polynucleotides can be single stranded.

In any of the embodiments herein, the alkyne or azido functional groups of the immobilized polynucleotides and the alkyne or azido functional groups on the barcode may not be protected prior to the irradiating step. In some embodiments, the functional groups may not be protected by a photo-sensitive group, moiety, or molecule prior to the irradiating step.

In any of the embodiments herein, the irradiation in step (b) can comprise using a photomask to selectively irradiate the second region of the immobilized polynucleotides.

In any of the embodiments herein, the method can further comprise capping the unreacted 3′ groups in the second region of the immobilized polynucleotides.

In any of the embodiments herein, the step (c) can be conducted for less than about 10 minutes. In some embodiments, the step (c) can be conducted for about 2 minutes to about 10 minutes. In some embodiments, the step (c) can be conducted for about 2 minutes to about 5 minutes.

In any of the embodiments herein, the irradiation intensity in step (c) can be between about 50 mW/cm² and about 100 mW/cm².

In any of the embodiments herein, the method can further comprise contacting the lawn of polynucleotides immobilized on the substrate with a second barcode sequence, wherein the second barcode sequence comprises an oligonucleotide with a 5′-azido or 5′-alkyne group that is a reaction partner for a Click reaction with the 3′-alkyne or 3′-azido group of the immobilized polynucleotides. In some embodiments, the second barcode sequence is different than the first barcode sequence. In some embodiments, the method further comprises masking a third region of the immobilized polynucleotides on the substrate such that only a fourth region of the immobilized polynucleotides on the substrate is capable of being exposed to light. In some embodiments, the third region comprises some of the polynucleotides in the first region. In some embodiments, the third region comprises the Click products formed in the second region.

In any of the embodiments herein, the method can further comprise irradiating the fourth region of immobilized polynucleotides on the substrate with a second ultraviolet light in the presence of a Cu (II) compound and a photoinitiator, thereby generating Cu(I), wherein the Cu(I) catalyzes the Click reaction between the 3′-alkyne or 3′-azido groups of the immobilized oligonucleotides or polynucleotides in the fourth region with the 5′-azido or 5′-alkyne groups of the second barcode sequence to generate a Click product comprising a 1,2,3-triazole group.

In some embodiments, the method can further comprise repeating steps (a)-(c) for N cycles, wherein N is an integer of 2 or greater. In some embodiments, the sequence of the barcode in each cycle can be different. In some embodiments, all of the immobilized oligonucleotides or polynucleotides have been converted to Click products after N Cycles.

In some embodiments, the method can comprise M rounds, M can be an integer of 2 or greater, and each of the M rounds can comprise one or more cycles. In some embodiments, M can be an integer of 4 or greater.

Also provided herein is a method for providing an array, comprising: (a) irradiating a substrate comprising an unmasked first region and a masked second region, whereby a photoresist in the first region can be degraded to render immobilized polynucleotide molecules in the first region available for hybridization and/or ligation, whereas immobilized polynucleotide molecules in the second region can be protected by a photoresist from hybridization and/or ligation, wherein polynucleotide molecules in the first and second regions can comprise a 3′-alkyne or 3′-azido group; (b) contacting the immobilized polynucleotide molecules in the first region with a first barcode sequence, wherein the first barcode sequence can comprise an oligonucleotide with a 5′-azido or 5′-alkyne group that is a reaction partner for a Click reaction with the 3′-alkyne or 3′-azido group of the immobilized polynucleotides in the first region, and wherein polynucleotide molecules in the second region may not receive the barcode sequence; (c) irradiating the first region of the substrate with a first ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the first region with the 5′-azido or 5′-alkyne groups of the first barcode sequence to generate a Click product comprising a 1,2,3-triazole group, thereby providing on the substrate an array comprising different polynucleotide molecules in the first and second regions. In some embodiments, the immobilized polynucleotides comprise a 3′-alkyne group. In some embodiments, the first barcode sequence comprises a 5′-azido group. In some embodiments, the immobilized polynucleotides comprise a 3′-azido group. In some embodiments, the first barcode sequence comprises a 5′-alkyne group.

In any of the embodiments herein, said contacting in step (b) can comprise flowing the first barcode sequence over the immobilized polynucleotides in the first region. In some embodiments, the method further comprises contacting the immobilized polynucleotides in the first region with a splint, wherein the splint comprises a first nucleotide sequence that is complementary to a nucleotide sequence on the immobilized polynucleotides in the first region. In some embodiments, the splint comprises a second nucleotide sequence that is complementary to a nucleotide sequence on the first barcode. In some embodiments, the immobilized polynucleotides in the first region are ligated to the first barcode using the splint as template. In some embodiments, the method further comprises removing the splint after the ligation.

In any of the embodiments herein, the Cu (II) compound can be copper sulfate. The Cu (II) compound is converted to Cu (I) in the first region following degradation of the photoresist. Cu (I) catalyzes the Click reaction between immobilized polynucleotides in the first region and the first barcode sequence. The immobilized polynucleotides protected by the photoresist do not undergo a Click reaction.

In any of the embodiments herein, the Cu (II) compound and the photoinitiator are flowed over the immobilized polynucleotides in the first region in a solution where the photoresist has been degraded. In some embodiments, the solution comprises DMSO.

In any of the embodiments herein, the alkyne or azido functional groups of the immobilized polynucleotides and the alkyne or azido functional groups on the barcode may not be protected prior to the irradiating step. In some embodiments, the functional groups are not protected by a photo-sensitive group, moiety, or molecule prior to the irradiating step.

In any of the embodiments herein, the irradiation in step (a) can comprise using a photomask to protect the photoresist in the second region and of the substrate and to selectively irradiate the first region of the substrate.

In any of the embodiments involving use of a photoresist, the steps (a)-(c) may be conducted for less than about 10 minutes. In some embodiments, the steps (a)-(c) may be conducted for about 2 minutes to about 10 minutes. In some embodiments, the steps (a)-(c) may be conducted for about 2 minutes to about 5 minutes.

In any of the embodiments herein, the irradiation intensity in step (c) may be between about 50 mW/cm² and about 100 mW/cm².

In any of the embodiments herein, the method can further comprise a step of providing the substrate, wherein the first and second regions have the same photoresist. In some embodiments, the providing step comprises applying the photoresist to the substrate, thereby forming a photoresist layer on the substrate. In some embodiments, the photoresist may be applied via spin coating. In some embodiments, the polynucleotide molecules on the substrate are embedded in the photoresist. In some embodiments, the polynucleotide molecules on the substrate are embedded in an underlayer, and the photoresist can form a photoresist layer on top of the underlayer. In some embodiments, the underlayer can be a soluble polymer.

In any of the embodiments herein, the photoresist in the first and/or second regions can comprise a photoacid generator. In some embodiments, the photoresist further comprises an acid scavenger. In some embodiments, the photoresist comprises a base quencher and/or a photosensitizer.

In any of the embodiments herein, the photoresist can further comprise a surfactant and/or a casting solvent.

In any of the embodiments herein, the substrate can be irradiated with a UV light. In any of the embodiments herein, the substrate can be irradiated through a patterned mask. In some embodiments, the method comprises removing the patterned mask after the irradiating step. In some embodiments, the same patterned mask can be re-used in a subsequent cycle of the irradiating and contacting steps, wherein the patterned mask is moved relative to the substrate. In some embodiments, a different patterned mask is used in a subsequent cycle of the irradiating and contacting steps.

In any of the embodiments herein, the photoresist in the first region of the substrate can be dissolved by a developer and removed.

Also provided herein is an array comprising a lawn of polynucleotides immobilized on a substrate, wherein the polynucleotides in different regions of the substrate are covalently linked to different barcode sequences via a 1,2,3-triazole group. In some embodiments, the 1,2,3-triazole linkages is formed from a Click reaction. In some embodiments, the Click reaction is catalyzed by Cu (I).

### BRIEF DESCRIPTION OF THE DRAWINGS

The drawings illustrate certain features and advantages of this disclosure. These embodiments are not intended to limit the scope of the appended claims in any manner.

FIG. 1 depicts an exemplary method for the in situ array generation using copper-catalyzed alkyne-azide click chemistry.

FIG. 2 is a schematic illustration of an exemplary method comprising M rounds, each of which comprise N cycles.

FIG. 3 shows an exemplary barcoded molecule generated using a method described herein.

FIG. 4 is an exemplary combinatorial method for in situ array generation using click chemistry.

### DETAILED DESCRIPTION

The practice of the techniques described herein may employ, unless otherwise indicated, conventional techniques and descriptions of organic chemistry, polymer technology, molecular biology (comprising recombinant techniques), cell biology, biochemistry, and sequencing technology, which are within the skill of those who practice in the art. Such conventional techniques comprise polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W. H. Freeman, New York N.Y.; Gait, “Oligonucleotide Synthesis: A Practical Approach” 1984, IRL Press, London; Nelson and Cox (2000), Lehninger, Principles of Biochemistry 3rd Ed., W. H. Freeman Pub., New York, N.Y.; and Berg et al. (2002) Biochemistry, 5th Ed., W. H. Freeman Pub., New York, N.Y., all of which are herein incorporated in their entirety by reference for all purposes.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In some aspects, provided herein is a method of patterning a surface in situ using one or more photomasks for producing an array on the surface, for example, by spatially-selective light-activated ligation (e.g., after hybridization to a splint) generating unique DNA sequences in unique spatial positions in the array. In any of the embodiments herein, the method disclosed herein can comprise a photo-hybridization ligation that is performed using any one or more elements of the exemplary methods and reagents described in US 2022/0228201 A1, US 2022/0228210 A1, and US 2022/0314187 A1, each of which is incorporated here by reference in its entirety for all purposes. For instance, some oligonucleotides on a substrate can be blocked (e.g., via photo-cleavable polymers, photo-cleavable moieties, or a photoresist) from hybridization and ligation while other oligonucleotides on the substrate can undergo hybridization and ligation to attach a nucleic acid barcode or part thereof. The deblocking can comprise: photo-cleaving a polymer that blocks an oligonucleotide in a prior cycle from hybridization and ligation; removing a photo-cleavable moiety of an oligonucleotide that blocks the oligonucleotide in a prior cycle from hybridization and ligation; or removing a photoresist that blocks the oligonucleotide in a prior cycle from hybridization and ligation.

High Resolution oligonucleotide arrays (e.g., ˜1.1 million spatial barcode diversity) for spatial transcriptomics may be made by multiple rounds of spatially selective light-activated de-protections and ligations, combinatorially generating unique DNA sequences in unique spatial positions (e.g., spots). In this method, a substrate (e.g., a glass slide) with immobilized photo-caged oligonucleotides undergoes the following steps: 1) spatially selective (e.g., by using a photo mask such as a patterned mask) photo-uncaging of photo-protective groups to create de-protected oligonucleotides, 2) ligation of the de-protected oligonucleotides to unique spatial barcodes (e.g., using a splint), 3) capping of unligated oligonucleotides at their 3′ ends by a terminal transferase, 4) changing the position of the photo mask (e.g., patterned mask) to spatially select a different set of oligonucleotides and repeating steps 1-3, 5) repeating steps 1-4 for N cycles for M rounds to achieve a desired barcode diversity. A method disclosed herein is compatible with and may comprise one or more base-by-base extension steps but does not depend on base-by-base extension.

One of the challenges with an enzymatic ligation approach is the relatively slow ligation reaction times (e.g., more than 1 hour). A secondary rate-limiting step of photocaged nucleotide extension is the multiple steps of de-protection and ligation, which depending on the light source can take on the order of for example, 5-15 minutes. Another challenge with the enzymatic ligation approach is overall low efficiency. In some embodiments, provided herein are methods for direct oligonucleotide photo-ligation by skipping the photo-deprotection step and directly ligating the oligonucleotides using photo-activated click chemistry reactions, as opposed to enzymatic ligation. In some embodiments, the method decouples light-activated de-protection and ligation into one simultaneous controlled chemical reaction. In some aspects, the method combines 1) a specific form of copper-dependent azide-alkyne click chemistry for DNA ligation that is polymerase read-through friendly, and 2) spatially-controlled light-induced (e.g., 365 nm) reduction of Cu(II) to Cu(I), which activates the click chemistry-based ligation.

In some aspects, a triazole 5′ azide and 3′ alkyne analog of natural phosphodiester DNA backbone can be formed by copper-based click chemistry. The ligation may or may not rely on DNA splint. In some aspects, the ligation product is polymerase read-through friendly and the arrays are amenable to reverse transcription enzymes in the downstream spatial transcriptomics workflow. The ligation product generated may be of a specific chemical structure of the DNA phosphate backbone, such as a synthetic triazole DNA analog that can serve as an appropriate substrate for thermostable polymerases. For instance, the DNA backbone may resemble a natural DNA phosphodiester bond comprising: 1) the absence of a rigid amide bond, and 2) the presence of a 3′-oxygen and 5′ methylene group as recognition sites for the DNA polymerase.

In some aspects, the method disclosed herein is a spatially specific Click chemistry reaction for DNA ligation. As seen with spatially-controlled crosslinking of a hydrogel via copper-based click chemistry (e.g., as described by Adzima B J, Tao Y, Kloxin C J, DeForest C A, Anseth K S, Bowman C N. Spatial and temporal control of the alkyne-azide cycloaddition by photoinitiated Cu(II) reduction. Nat Chem. 2011 March;3(3):256-59, and Adzima, B. J. and Bowman, C. N. The emerging role of click reactions in chemical and biological engineering. AIChE J. 2012, 58: 2952-65, the contents of each of which are herein incorporated by reference in their entireties), the speed of the reaction can easily be controlled by the dose of the UV light, specific to the optical setup.

Provided herein is a method for providing an array, comprising: (a) contacting a lawn of polynucleotides immobilized on a substrate with a first barcode sequence, wherein the polynucleotides comprise a 3′-alkyne or 3′-azido group, and wherein the first barcode sequence comprises an oligonucleotide with a 5′-azido or 5′-alkyne group that is a reaction partner for a Click reaction with the 3′-alkyne or 3′-azido group of the immobilized polynucleotides; (b) masking a first region of the immobilized polynucleotides on the substrate such that only a second region of the immobilized polynucleotides on the substrate are capable of being exposed to light; (c) irradiating the second region of immobilized polynucleotides on the substrate with a first ultraviolet light in the presence of a Cu (II) compound and a photoinitiator, thereby generating Cu(I), wherein the Cu(I) may catalyze the Click reaction between the 3′-alkyne or 3′-azido groups of the immobilized polynucleotides in the second region with the 5′-azido or 5′-alkyne groups of the first barcode sequence to generate a Click product comprising a 1,2,3-triazole group, thereby providing on the substrate an array comprising the first and second regions of the immobilized polynucleotides, wherein the second region of immobilized polynucleotides are barcoded with the first barcode sequence and the second region of immobilized polynucleotides are not be barcoded with the first barcode sequence. In some embodiments, the immobilized polynucleotides comprises a 3′-alkyne group. In some embodiments, the first barcode sequence comprises a 5′-azido group. In some embodiments, the immobilized polynucleotides comprises a 3′-azido group. In some embodiments, the first barcode sequence comprises a 5′-alkyne group.

In some aspects, the method uses splinted photo-click chemistry in in situ array synthesis. In some aspects, the method disclosed herein combines light-activated de-protection and ligation into a single step. The method offers an advantage of having a one-step photoligation method instead of a two-step photo-deprotection and ligation method. Such a single step method of mask photolithography mitigates risk of DNA damage caused by excessive UV dosage. The substrate (e.g., a glass slide) contains a lawn of DNA polynucleotides (e.g., immobilized polynucleotides) containing 3′-alkyne (or 3′-azido) groups available for Click-chemistry based ligation. The immobilized polynucleotides are complementary to a specific splint (e.g., splint adapter sequence). An excess of double stranded DNA containing an overhang with a complementary sequence to the lawn polynucleotides and a spot-specific (e.g., position-specific) barcode bearing a 5′-azido (or 5′-alkyne) group are flowed over the slide surface. A large number of barcodes (e.g., spot-specific barcodes) will be hybridized to the available immobilized polynucleotides. Upon UV illumination (e.g., irradiation with light of ˜365 nm) in the presence of copper a Cu (II) compound (e.g., copper sulfate) and a photoinitiator (e.g., Irgacure 2959) the barcodes are ligated to the immobilized polynucleotides. One cycle of the hybridization and ligation reaction can take on the order of a few minutes, depending on the optical setup. In some embodiments, the method further comprises capping the unreacted 3′ groups of the immobilized polynucleotides. One cycle of hybridization and ligation is followed by a capping reaction to remove unreacted 3′ groups. The capping step (e.g., with a monovalent PEG-azide) at the end of each round of ligation removes unreacted available alkyne or azido groups. It is possible that single DNA polynucleotides may not all completely undergo the successive ligation reactions, leaving available alkyne groups on the surface that may nonspecifically react with polynucleotides introduced in future ligation rounds. Therefore, a capping step is performed at the end of each ligation round to remove unreacted available alkyne groups. The capping step ensures that the ligation reactions are complete. The photomask (e.g., patterned mask) is then moved to a new position for another cycle of hybridization, ligation, and capping. The cycles of hybridization and ligation are repeated in M rounds, where each round comprises N cycles to achieve a desired barcode diversity up to N^{M}.

In some embodiments, the irradiation dose is tuned by varying the selection of the optical setup, including the power of the UV lamp and duration of irradiation. For example, the choice of a mask aligner, which provides focused illumination under an objective lens would provide much greater UV intensity than a un-focused simple benchtop lamp, thereby potentially reducing the time (e.g., from 50 minutes to 6.7 minutes). For example, a high-power mask aligner can provide an irradiance of 75 mW/cm2, which is 7.5× more powerful than the un-focused simple benchtop lamp.

In some embodiments, the method further comprises contacting the lawn of polynucleotides with a free radical scavenger. The choice and concentration of the free radical-scavenger is to prevent non-specific damage, scission of the nucleic acids, or oxidative DNA damage mediated by the copper. For example, dimethyl sulphoxide (DMSO; 10%) can prevent the incidence of DNA breakage from copper-related scission by greater than 90%.

In some embodiments, the method comprises contacting the lawn of immobilized polynucleotides with a splint. The length and specificity of the splint can be optimized to prevent un-splinted 5′ end-to-3′ end ligations.

Also provided herein is a method for providing an array, comprising: (a) irradiating a substrate comprising an unmasked first region and a masked second region, whereby a photoresist in the first region is degraded to render immobilized polynucleotide molecules in the first region available for hybridization and/or ligation, whereas immobilized polynucleotide molecules in the second region are protected by a photoresist from hybridization and/or ligation, wherein polynucleotide molecules in the first and second regions comprise a 3′-alkyne or 3′-azido group; (b) contacting the immobilized polynucleotide molecules in the first region with a first barcode sequence, wherein the first barcode sequence comprises an oligonucleotide with a 5′-azido or 5′-alkyne group that is a reaction partner for a Click reaction with the 3′-alkyne or 3′-azido group of the immobilized polynucleotides in the first region, and wherein polynucleotide molecules in the second region do not receive the barcode sequence; (c) irradiating the first region of the substrate with a first ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the first region with the 5′-azido or 5′-alkyne groups of the first barcode sequence to generate a Click product comprising a 1,2,3-triazole group, thereby providing on the substrate an array comprising different polynucleotide molecules in the first and second regions. In some aspects, a copper-mediated azide-alkyne coupling is used as part of the photoresist. The photoresist can be removed to unveil polynucleotides with 3′ alkynes which can be conjugated with a 5′ azide oligonucleotide. In some embodiments, the method further comprises contacting the immobilized polynucleotides in the first region with a splint, wherein the splint comprises a first nucleotide sequence that is complementary to a nucleotide sequence on the immobilized polynucleotides in the first region. The use of a splint can greatly accelerate the reaction kinetics.

Also provided herein is an array comprising a lawn of polynucleotides immobilized on a substrate, wherein the polynucleotides in different regions of the substrate are covalently linked to different barcode sequences via a 1,2,3-triazole group.

### II. Molecular Arrays and Photolithography

### A. Molecular Arrays.

In some aspects, the method provided herein comprises attaching oligonucleotides (e.g. a barcode) to a substrate. Oligonucleotides may be attached to the substrate according to the methods set forth in U.S. Pat. Nos. 6,737,236, 7,259,258, 7,375,234, 7,427,678, 5,610,287, 5,807,522, 5,837,860, and 5,472,881; U.S. Patent Application Publication Nos. 2008/0280773 and 2011/0059865; Shalon et al. (1996) Genome Research, 639-645; Rogers et al. (1999) Analytical Biochemistry 266, 23-30; Stimpson et al. (1995) Proc. Natl. Acad. Sci. USA 92, 6379-6383; Beattie et al. (1995) Clin. Chem. 45, 700-706; Lamture et al. (1994) Nucleic Acids Research 22, 2121-2125; Beier et al. (1999) Nucleic Acids Research 27, 1970-1977; Joos et al. (1997) Analytical Biochemistry 247, 96-101; Nikiforov et al. (1995) Analytical Biochemistry 227, 201-209; Timofeev et al. (1996) Nucleic Acids Research 24, 3142-3148; Chrisey et al. (1996) Nucleic Acids Research 24, 3031-3039; Guo et al. (1994) Nucleic Acids Research 22, 5456-5465; Running and Urdea (1990) BioTechniques 8, 276-279; Fahy et al. (1993) Nucleic Acids Research 21, 1819-1826; Zhang et al. (1991) 19, 3929-3933; and Rogers et al. (1997) Gene Therapy 4, 1387-1392. The entire contents of each of the foregoing documents are incorporated herein by reference.

In some embodiments, a substrate comprising an array of molecules is provided, e.g., in the form of a lawn of polymers (e.g., polynucleotides) on the substrate in a pattern. Examples of polymers on an array may include, but are not limited to, nucleic acids, peptides, phospholipids, polysaccharides, heteromacromolecules in which a known drug is covalently bound to any of the above, polyurethanes, polyesters, polycarbonates, polyureas, polyamides, polyethyleneimines, polyarylene sulfides, polysiloxanes, polyimides, and polyacetates. The molecules occupying different features of an array typically differ from one another, although some redundancy in which the same polymer occupies multiple features can be useful as a control. For example, in a nucleic acid array, the nucleic acid molecules within the same feature are typically the same, whereas nucleic acid molecules occupying different features are mostly different from one another.

In some examples, the molecules on the array are nucleic acids. The nucleic acid molecule can be single-stranded or double-stranded. Nucleic acid molecules on an array may be DNA or RNA. The DNA may be single-stranded or double-stranded. The DNA may include, but are not limited to, mitochondrial DNA, cell-free DNA, complementary DNA (cDNA), genomic DNA, plasmid DNA, cosmid DNA, bacterial artificial chromosome (BAC), or yeast artificial chromosome (YAC). The RNA may include, but are not limited to, mRNAs, tRNAs, snRNAs, rRNAs, retroviruses, small non-coding RNAs, microRNAs, polysomal RNAs, pre-mRNAs, intronic RNA, viral RNA, cell free RNA and fragments thereof. The non-coding RNA, or ncRNA can include snoRNAs, microRNAs, siRNAs, piRNAs and long nc RNAs.

Arrays can be prepared by a variety of methods. In some embodiments, arrays are prepared through the synthesis (e.g., in situ synthesis) of polynucleotides on the array, or by jet printing, or lithography. In some embodiments, an array is prepared by contacting (e.g., flowing over) a lawn of polynucleotides immobilized on a substrate with barcoded oligonucleotides (e.g., oligonucleotides comprising barcode sequences). In some embodiments, the immobilized polynucleotides on the lawn comprise a 3′-alkyne or 3′-azido group, and the barcoded oligonucleotides (e.g., oligonucleotides comprising barcode sequences) comprise a 5′-azido or 5′-alkyne group. The 5′-azido or 5′-alkyne groups are reaction partners for a Click chemistry reaction with the 3′-alkyne or 3′-azido groups of the immobilized polynucleotides.

In some embodiments, the polynucleotides immobilized on the substrate are identical in sequence. The polynucleotides within a same feature are typically the same, whereas the polynucleotides occupying different features are mostly different from one another. In some aspects, the polynucleotides immobilized on the substrate are of varied lengths. In some embodiments, the polynucleotides length is about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, about 40, about 45, about 50 in length. In some embodiments, the polynucleotides immobilized on substrate prior are between about 4 and about 50 nucleotides in length. In some embodiments, the polynucleotides are double stranded. In some embodiments, the polynucleotides are single stranded.

In some aspects, the polynucleotides (e.g., immobilized polynucleotides or barcoded polynucleotides) comprise functional groups. In some aspects, the functional groups of the polynucleotides are alkyne or azido functional groups. The functional groups on the polynucleotides can be protected or unprotected. In some embodiments, the functional groups are not protected by a photo-sensitive group, moiety, or molecule prior to the irradiating step.

In some embodiments, the molecules (e.g., polynucleotides) on an array comprise barcodes. A barcode sequence can be of varied length. In some embodiments, the barcode sequence is about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about or about 70 nucleotides in length. In some embodiments, the barcode sequence is between about 4 and about 25 nucleotides in length. In some embodiments, the barcode sequences is between about 10 and about 50 nucleotides in length. The nucleotides can be completely contiguous, i.e., in a single stretch of adjacent nucleotides, or they can be separated into two or more separate subsequences that are separated by 1 or more nucleotides. In some embodiments, the barcode sequence can be about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or longer. In some embodiments, the barcode sequence can be at least about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or longer. In some embodiments, the barcode sequence can be at most about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or shorter.

The polynucleotide can include one or more (e.g., two or more, three or more, four or more, five or more) Unique Molecular Identifiers (UMIs). A unique molecular identifier is a contiguous nucleic acid segment or two or more non-contiguous nucleic acid segments that function as a label or identifier for a particular analyte, or for a capture probe that binds a particular analyte (e.g., via the capture domain).

A UMI can be unique. A UMI can include one or more specific polynucleotides sequences, one or more random nucleic acid and/or amino acid sequences, and/or one or more synthetic nucleic acid and/or amino acid sequences.

In some embodiments, the UMI is a nucleic acid sequence that does not substantially hybridize to analyte nucleic acid molecules in a biological sample. In some embodiments, the UMI has less than 90% sequence identity (e.g., less than 80%, 70%, 60%, 50%, or less than 40% sequence identity) to the nucleic acid sequences across a substantial part (e.g., 80% or more) of the nucleic acid molecules in the biological sample.

The UMI can include from about 6 to about 20 or more nucleotides within the sequence of capture probes, e.g., barcoded polynucleotides in an array generated using a method disclosed herein. In some embodiments, the length of a UMI sequence can be about 6, 7, 8, 9, 10, 11, 12, 13, 14, 15, 16, 17, 18, 19, 20 nucleotides or longer. In some embodiments, the length of a UMI sequence can be at least about 6, 7, 8, 9, 10, 11, 12, 13, 14, 15, 16, 17, 18, 19, 20 nucleotides or longer. In some embodiments, the length of a UMI sequence is at most about 6, 7, 8, 9, 10, 11, 12, 13, 14, 15, 16, 17, 18, 19, 20 nucleotides or shorter. These nucleotides can be contiguous, i.e., in a single stretch of adjacent nucleotides, or they can be separated into two or more separate subsequences that are separated by 1 or more nucleotides. Separated UMI subsequences can be from about 4 to about 16 nucleotides in length. In some embodiments, the UMI subsequence can be about 4, 5, 6, 7, 8, 9, 10, 11, 12, 13, 14, 15, 16 nucleotides or longer. In some embodiments, the UMI subsequence can be at least about 4, 5, 6, 7, 8, 9, 10, 11, 12, 13, 14, 15, 16 nucleotides or longer. In some embodiments, the UMI subsequence can be at most about 4, 5, 6, 7, 8, 9, 10, 11, 12, 13, 14, 15, 16 nucleotides or shorter.

In some embodiments, a UMI is attached to other parts of the nucleotide in a reversible or irreversible manner. In some embodiments, a UMI is added to, for example, a fragment of a DNA or RNA sample before, during, and/or after sequencing of the analyte. In some embodiments, a UMI allows for identification and/or quantification of individual sequencing-reads. In some embodiments, a UMI is a used as a fluorescent barcode for which fluorescently labeled oligonucleotide probes hybridize to the UMI.

In some embodiments, a method provided herein further comprises a step of providing the substrate. A wide variety of different substrates can be used for the foregoing purposes. In general, a substrate can be any suitable support material. The substrate may comprise materials of one or more of the IUPAC Groups 4, 6, 11, 12, 13, 14, and 15 elements, plastic material, silicon dioxide, glass, fused silica, mica, ceramic, or metals deposited on the aforementioned substrates. Exemplary substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene and polycarbonate.

A substrate can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip or a slide such as a microscope slide).

Provided herein is a method for providing an array comprising contacting a lawn of polynucleotides immobilized on a substrate with a first barcode sequence, wherein the lawn of polynucleotides comprises a 3′-alkyne or 3′-azido group, and wherein the first barcode comprises an oligonucleotide with a 5′-azido or 5′-alkyne group that is a reaction partner for a Click reaction with the 3′-alkyne or 3′-azido group of the immobilized polynucleotides. The method comprises providing on the substrate an array comprising a first and second region of the immobilized polynucleotides, wherein the second region of immobilized polynucleotides is barcoded with the first barcode sequence and the first region of immobilized polynucleotides is not barcoded with the first barcode sequence.

### B. Photolithography

Disclosed herein are methods of masking (e.g., using a photomask) and spatially irradiating a region on the array. These methods are performed after the steps disclosed in Section II.A. Disclosed herein is a method for providing an array, comprising: (a) contacting a lawn of polynucleotides immobilized on a substrate with a first barcode sequence, wherein the lawn of polynucleotides comprises a 3′-alkyne or 3′-azido group, and wherein the first barcode sequence comprises an oligonucleotide with a 5′-azido or 5′-alkyne group that is a reaction partner for a Click reaction with the 3′-alkyne or 3′-azido group of the immobilized polynucleotides; and (b) masking a first region of the immobilized oligonucleotides or polynucleotides on the substrate such that only a second region of the immobilized polynucleotides on the substrate are capable of being exposed to light.

In some aspects, provided herein is a method of patterning a surface in situ for producing an array on the surface. In some embodiments, the method comprises assembling barcode sequences on immobilized oligonucleotides, e.g., based on hybridization and ligation, on a slide surface. In some embodiments, the in situ method uses photolithography to enable barcodes to be generated selectively on a discrete location on a slide surface. Hybridization and ligation of barcodes can be controlled, for example, using a contact photolithography process. For example, ligation can be achieved by exposing polynucleotides for ligation by spatially irradiating a region on the substrate through a photomask.

In some embodiments, arrays are prepared via photolithographic synthesis, wherein functional groups (e.g., azide or alkyne groups on the oligonucleotides) are modified using a photolithographic mask (applied to specific areas of the substrate) and light, thereby producing an array having spatially controlled ligation. In some embodiments, the method comprises using a photomask to selectively irradiate a region (e.g., a first or second region) of the immobilized polynucleotides, while another region (e.g., first or second region) remains unexposed.

The mask is designed in such a way that the exposure sites can be selected, and thus specify the coordinates on the array where each polynucleotide (e.g., barcode) can be attached. The process can be repeated, a new mask is applied activating different sets of sites and coupling different polynucleotides, allowing arbitrary polynucleotides to be constructed at each site. This process can be used to synthesize hundreds of thousands or millions of different polynucleotides. In some embodiments, the substrate is irradiated through a patterned mask. The mask may be an opaque plate or film with transparent areas that allow light to shine through in a pre-defined pattern. After the irradiation step, the mask may be removed, translated to a different region on the substrate, or rotated. In some embodiments, a different photomasking pattern may be used in each barcoding round. In some embodiments, the same photomasking pattern may be used in each barcoding round.

The material of the photomask used herein may comprise silica with chrome in the opaque part. For example, the photomask may be transparent fused silica blanks covered with a pattern defined with a chrome metal absorbing film. The photomask may be used at various irradiation wavelengths, which include but are not limited to, 365 nm, 248 nm, and 193 nm. In some embodiments, the irradiation step herein can be performed for a duration of between about 1 minute and about 10 minutes, for example, for about 2 minutes, about 4 minutes, about 6 minutes, or about 8 minutes. In some embodiments, the irradiation can be performed at a total light dose of between about one and about ten mW/mm², for example, at about 2 mW/mm², about 4 mW/mm², about 6 mW/mm², or about 8 mW/mm². In some embodiments, the irradiation can be performed at a total light dose of between about one and about ten mW/mm², and for a duration of between about 1 minute and about 10 minute and about ten mW/mm² and for a duration of between about 1 minute and about 10 minutes.

### i) Photoresist

A photoresist is a light-sensitive material used in processes (such as photolithography and photoengraving) to form a pattern on a surface. A photoresist may comprise a polymer, a sensitizer, and/or a solvent. The photoresist composition used herein is not limited to any specific proportions of the various components.

Photoresists can be classified as positive or negative. In positive photoresists, the photochemical reaction that occurs during light exposure weakens the polymer, making it more soluble to developer, so a positive pattern is achieved. In the case of negative photoresists, exposure to light causes polymerization of the photoresist, and therefore the negative photoresist remains on the surface of the substrate where it is exposed, and the developer solution removes only the unexposed areas. In some embodiments, the photoresist used herein is a positive photoresist. In some embodiments, the photoresist is removable with UV light.

In some embodiments, provided herein is a method for providing an array, comprising: (a) irradiating a substrate comprising an unmasked first region and a masked second region, whereby a photoresist in the first region is degraded to render immobilized polynucleotide molecules in the first region available for hybridization and/or ligation, whereas immobilized polynucleotide molecules in the second region are protected by a photoresist from hybridization and/or ligation, wherein polynucleotide molecules in the first and second regions comprise a 3′-alkyne or 3′-azido group. In some embodiments, the method further comprises a step of providing the substrate, wherein the first and second regions have the same photoresist.

The photoresist may experience changes in pH upon irradiation. In some embodiments, the photoresist in the first region comprises a photoacid generator (PAG). In some embodiments, the photoresist in the second region comprises a photoacid generator. In some embodiments, the photoresist in the first and the second region comprises a photoacid generator. In some embodiments, the photoresist in the first and the second region comprises the same photoacid generator. In some embodiments, the photoresist in the first and the second region comprises different photoacid generators. In some embodiments, the photoacid generator or photoacid generators irreversibly release protons upon absorption of light. Photoacid generators may be used as components of photocurable polymer formulations and chemically amplified photoresists. Examples of photoacid generators include triphenylsulfonium triflate, diphenylsulfonium triflate, diphenyliodonium nitrate, N-Hydroxynaphthalimide triflate, triarylsulfonium hexafluorophosphate salts, N-hydroxy-5-norbornene-2,3-dicarboximide perfluoro-1-butanesulfonate, bis(4-tert-butylphenyl)iodonium perfluoro-1-butanesulfonate, etc.

In some embodiments, the photoresist further comprises an acid scavenger. In some embodiments, the photoresist in the first and the second region comprises the same acid scavenger. In some embodiments, the photoresist in the first and the second region comprises different acid scavengers. In some embodiments, an acid scavenger acts to neutralize, adsorb and/or buffer acids, and may comprise a base or alkaline compound. In some embodiments, acid scavengers act to reduce the amount or concentration of protons or protonated water. In some embodiments, an acid scavenger acts to neutralize, diminish, or buffer acid produced by a photoacid generator. In some embodiments, an acid scavenger exhibits little or no stratification over time or following exposure to heat. In some embodiments, acid scavengers may be further subdivided into “organic bases” and “polymeric bases.” A polymeric base is an acid scavenger (e.g., basic unit) attached to a longer polymeric unit. A polymer is typically composed of a number of coupled or linked monomers. The monomers can be the same (to form a homopolymer) or different (to form a copolymer). In a polymeric base, at least some of the monomers act as acid scavengers. An organic base is a base which is joined to or part of a non-polymeric unit. Non-limiting examples of organic bases include, without limitation, amine compounds (e.g., primary, secondary and tertiary amines). Generally any type of acid scavenger, defined here as a traditional Lewis Base, an electron pair donor, can be used in accordance with the present disclosure.

In some embodiments, the photoresist further comprises a base quencher. Base quenchers may be used in photoresist formulations to improve performance by quenching reactions of photoacids that diffuse into unexposed regions. Base quenchers may comprise aliphatic amines, aromatic amines, carboxylates, hydroxides, or combinations thereof. Examples of base quenchers include but are not limited to, trioctylamine, 1,8-diazabicyclo[5.4.0]undec-7-ene (DBU), 1-piperidineethanol (1PE), tetrabutylammonium hydroxide (TBAH), dimethylamino pyridine, 7-diethylamino-4-methyl coumarin (Coumarin 1), tertiary amines, sterically hindered diamine and guanidine bases such as 1,8-bis(dimethylamino)naphthalene (PROTON SPONGE), berberine, or polymeric amines such as in the PLURONIC or TETRONIC series commercially available from BASF. In some embodiments, the photoresist in the first and the second region comprises the same base quencher. In some embodiments, the photoresist in the first and the second region comprises different base quenchers.

In some embodiments, the photoresist further comprises a photosensitizer. A photosensitizer is a molecule that produces a chemical change in another molecule in a photochemical process. Photosensitizers are commonly used in polymer chemistry in reactions such as photopolymerization, photocrosslinking, and photodegradation. Photosensitizers generally act by absorbing ultraviolet or visible region of electromagnetic radiation and transferring it to adjacent molecules. In some embodiments, photosensitizer shifts the photo sensitivity to a longer wavelength of electromagnetic radiation. The sensitizer, also called a photosensitizer, is capable of activating the PAG at, for example, a longer wavelength of light in accordance with an aspect of the present invention. Preferably, the concentration of the sensitizer is greater than that of the PAG, such as 1.1 times to 5 times greater, for example, 1.1 times to 3 times greater the concentration of PAG. Exemplary sensitizers suitable for use in the invention include but are not limited to, isopropylthioxanthone (ITX) and 10H-phenoxazine (PhX). In some embodiments, the photoresist in the first and the second region comprises the same photosensitizer. In some embodiments, the photoresist in the first and the second region comprises different photosensitizers.

In some embodiments, the photoresist further comprises a matrix. The matrix generally refers to polymeric materials that may provide sufficient adhesion to the substrate when the photoresist formulation is applied to the top surface of the substrate, and may form a substantially uniform film when dissolved in a solvent and spread on top of a substrate. Examples of a matrix may include, but are not limited to, polyester, polyimide, polyethylene naphthalate (PEN), polyvinyl chloride (PVC), polymethylmethacrylate (PMMA) and polycarbonate, or a combination thereof. The matrix may be chosen based on the wavelength of the radiation used for the generation of acid when using the photoresist formulation, the adhesion properties of the matrix to the top surface of the substrate, the compatibility of the matrix to other components of the formulation, and the ease of removable or degradation (if needed) after use. In some embodiments, the photoresist in the first and the second region comprises the same matrix. In some embodiments, the photoresist in the first and the second region comprises different matrices.

In some embodiments, the photoresist further comprises a surfactant. Surfactants may be used to improve coating uniformity, and may include ionic, non-ionic, monomeric, oligonucleotidemeric, and polymeric species, or combinations thereof. Examples of possible surfactants include fluorine-containing surfactants such as the FLUORAD series available from 3M Company in St. Paul, Minn., and siloxane-containing surfactants such as the SILWET series available from Union Carbide Corporation in Danbury, Conn. In some embodiments, the photoresist in the first and the second region comprises the same surfactant. In some embodiments, the photoresist in the first and the second region comprises different surfactants.

In some embodiments, the photoresist further comprises a casting solvent. A casting solvent may be used so that the photoresist may be applied evenly on the substrate surface to provide a defect-free coating. Examples of suitable casting solvents may include ethers, glycol ethers, aromatic hydrocarbons, ketones, esters, ethyl lactate, γ-butyrolactone, cyclohexanone, ethoxyethylpropionate (EEP), a combination of EEP and gamma-butyrolactone (GBL), and propylene glycol methyl ether acetate (PGMEA). In some embodiments, the photoresist in the first and the second region comprises the same casting solvent. In some embodiments, the photoresist in the first and the second region comprises different casting solvents.

Methods of applying photoresist to the substrate include, but are not limited to, dipping, spreading, spraying, or any combination thereof. In some embodiments, the photoresist is applied via spin coating, thereby forming a photoresist layer on the substrate.

In some embodiments, the photoresist is in direct contact with the polynucleotides on the substrate. In some embodiments, the polynucleotide molecules on the substrate are embedded in the photoresist. In some embodiments, the photoresist is not in direct contact with the polynucleotides. In some embodiments, polynucleotide molecules on the substrate are embedded in an underlayer that is underneath the photoresist. For example, polynucleotide molecules on the substrate may be embedded in a soluble polymer underlayer (e.g., a soluble polyimide underlayer (XU-218)), and the photoresist forms a photoresist layer on top of the underlayer.

In some embodiments, the photoresist may be removed and re-applied. For example, the photoresist may be stripped from the substrate and/or the polynucleotides ligated to the substrate. Removal of photoresist can be accomplished with various degrees of effectiveness. In some embodiments, the photoresist is completely removed from the substrate and/or the polynucleotides ligated to the substrate before re-application. Methods of removing photoresist may include, but are not limited to, using organic solvent mixtures, using liquid chemicals, exposure to a plasma environment, or other dry techniques such as UV/O3 exposure. In some embodiments, the photoresist is stripped using organic solvent. In some embodiments, the photoresist in a region of the substrate (e.g., first and/or second region) is dissolved by a developer and removed. In some embodiments, one or more photomasks may be used to selectively remove photoresist on the substrate.

Using a series of photomasks, photoresist in desired regions of the substrate may be iteratively irradiated and subsequently removed.

The material of the photomask used herein may comprise silica with chrome in the opaque part. For example, the photomask may be transparent fused silica blanks covered with a pattern defined with a chrome metal absorbing film. The photomask may be used at various irradiation wavelengths, which include but are not limited to, 365 nm, 248 nm, and 193 nm.

In some embodiments, the method comprises irradiating a substrate covered with a photoresist. In some embodiments, the irradiation is selective, for example, where one or more photomasks can be used such that only one or more specific regions of the array are exposed to irradiation stimuli (e.g., exposure to light such as UV, and/or exposure to heat induced by laser). In some embodiments, the substrate comprises an unmasked region (e.g., unmasked second region) and a masked region (e.g., masked first region). In some embodiments, the photoresist in the unmasked region is degraded upon irradiation to render polynucleotide molecules available for hybridization and/or ligation. In some embodiments, the polynucleotides in the masked region are protected by a photoresist. In some embodiments, the photoresist is a positive photoresist. For example, the photoresist in a region (e.g., second region) is exposed to light and degraded when the photoresist in the different region (e.g., first region) is photomasked. In some embodiments, the method further comprises attaching an polynucleotide comprising a barcode sequence to polynucleotide molecules in the unmasked region (e.g., unmasked second region) via hybridization and ligation, while polynucleotide molecules in a different region (e.g., masked first region) do not receive the barcode sequence. A method for providing an array, comprising: (a) irradiating a substrate comprising an unmasked first region and a masked second region, whereby a photoresist in the first region is degraded to render immobilized polynucleotide molecules in the first region available for hybridization and/or ligation, whereas immobilized polynucleotide molecules in the second region are protected by a photoresist from hybridization and/or ligation, wherein polynucleotide molecules in the first and second regions comprise a 3′-alkyne or 3′-azido group; (b) contacting the immobilized polynucleotide molecules in the first region with a first barcode sequence, wherein the first barcode sequence comprises an oligonucleotide with a 5′-azido or 5′-alkyne group that is a reaction partner for a Click reaction with the 3′-alkyne or 3′-azido group of the immobilized polynucleotides in the first region, and wherein polynucleotide molecules in the second region do not receive the barcode sequence; (c) irradiating the first region of the substrate with a first ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the first region of the substrate with the 5′-azido or 5′-alkyne groups of the first barcode sequence to generate a Click product comprising a 1,2,3-triazole group, thereby providing on the substrate an array comprising different polynucleotide molecules in the first and second regions.

### III. Hybridization and Click Chemistry-Based DNA Ligation

Disclosed herein are methods for hybridizing and ligating polynucleotides on the array (e.g., ligating the immobilized polynucleotides to the barcoded polynucleotides). The polynucleotides (e.g., immobilized polynucleotides) described herein may be linked to the barcoded probe sequences via triazole groups formed through a light activated click chemistry reaction, as described herein

### A. Hybridization

In some embodiments, the method comprises contacting the lawn of immobilized polynucleotides with a splint. A splint is an oligonucleotide that, when hybridized to other oligonucleotide or polynucleotides, acts as a “splint” to position the oligonucleotides or polynucleotides next to one another so that they can be ligated together. In some embodiments, the splint is DNA or RNA. The splint can include a nucleotide sequence that is partially complimentary to nucleotide sequences from two or more different oligonucleotides or polynucleotides. In some embodiments, the splint assists in ligating a “donor” oligonucleotide (e.g., barcoded barcode sequence) and an “acceptor” oligonucleotide (e.g., immobilized oligonucleotides or polynucleotides).

Splints have been described, for example, in US20150005200A1. A splint may be used for ligating two polynucleotides. The sequence of a splint may be configured to be in part complementary to at least a portion of the immobilized polynucleotides that are attached to the substrate and in part complementary to at least a portion of the barcoded polynucleotides (e.g., first barcode sequence). In one case, the splint can hybridize to the immobilized polynucleotide via its complementary sequence; once hybridized, the barcoded polynucleotides (e.g., first barcode sequence) or polynucleotide segment of the splint can then be attached to the immobilized polynucleotide attached to the substrate via any suitable attachment mechanism, such as, for example, a ligation reaction via Click chemistry. The splint complementary to both the immobilized polynucleotide and the barcoded polynucleotide can then be denatured (or removed) with further processing. The method comprises removing the splint after ligation. The method of attaching the barcoded polynucleotides to the immobilized polynucleotide can then be optionally repeated to ligate a third, and/or a fourth, and/or more parts of the barcode sequence onto the array with the aid of splint(s). In some embodiments, the splint is between 10 and 50 oligonucleotides in length, e.g., between 10 and 45, 10 and 40, 10 and 35, 10 and 30, 10 and 25, or 10 and 20 oligonucleotides in length. In some embodiments, the splint is between 15 and 50, 15 and 45, 15 and 40, 15 and 35, 15 and 30, 15 and 30, or 15 and 25 nucleotides in length.

In some embodiments, the splint comprises a sequence that is complementary to an polynucleotide (e.g., an immobilized oligonucleotide), or a portion thereof, and a sequence that is complementary to a barcoded polynucleotide containing a barcode sequence, or a portion thereof. In some embodiments, the splint comprises a sequence that is perfectly complementary (e.g., is 100% complementary) to an polynucleotide (e.g., an immobilized polynucleotide), or a portion thereof, and/or a sequence that is perfectly complementary to an polynucleotide containing a barcode, or a portion thereof. In some embodiments, the splint comprises a sequence that is not perfectly complementary (e.g., is not 100% complementary) to an polynucleotide (e.g., an immobilized polynucleotide), or a portion thereof, and/or a sequence that is not perfectly complementary to an polynucleotide containing a barcode, or a portion thereof. In some embodiments, the splint comprises a sequence that is at least 70%, 75%, 80%, 85%, 90%, 95%, 96%, 97%, 98%, or 99% complementary to an polynucleotide (e.g., an immobilized polynucleotide), or a portion thereof, and/or a sequence that is complementary to an polynucleotide containing a barcode, or a portion thereof. In some embodiments, the splint comprises a sequence that is perfectly complementary (e.g., is 100% complementary) to an polynucleotide (e.g., an immobilized polynucleotide), or a portion thereof, but is not perfectly complementary to a sequence that is complementary to an polynucleotide containing a barcode, or a portion thereof. In some embodiments, the splint comprises a sequence that is not perfectly complementary (e.g., is not 100% complementary) to an polynucleotide (e.g., an immobilized oligonucleotide), or a portion thereof, but is perfectly complementary to a sequence that is complementary to an polynucleotide containing a barcode, or a portion thereof. So long as the splint is capable of hybridizing to an polynucleotide (e.g., an immobilized polynucleotide), or a portion thereof, and to a sequence that is complementary to a barcoded polynucleotide containing a barcode sequence, or a portion thereof, the splint need not have a sequence that is perfectly complementary to either the polynucleotide (e.g., the immobilized polynucleotide) or to the a polynucleotide containing a barcode.

In some embodiments, the method for providing an array described herein comprises contacting the lawn of immobilized polynucleotides with a splint, wherein the splint comprises a first nucleotide sequence that is complementary to a nucleotide sequence on the immobilized polynucleotides. In some embodiments, a first splint hybridizes to immobilized polynucleotides in a second region. In some embodiments, an barcoded polynucleotide is not ligated to immobilized polynucleotides in the first region. In some embodiments, the hybridization region between the first splint and the polynucleotides (e.g., immobilized polynucleotides and/or barcoded polynucleotides) is at least 3, 4, 5, 6, 7, 8, 9, 10 base pairs (bp) or more than 10 bp. In some embodiments, the hybridization region between the first splint and the polynucleotides (e.g., immobilized polynucleotides) in the second region is at least 3, 4, 5, 6, 7, 8, 9, 10 bp or more than 10 bp.

In some embodiments, the polynucleotide is ligated using the splint as template without gap filling prior to the ligation. In some embodiments, the polynucleotide is ligated using the splint as template with gap filling prior to the ligation. In some embodiments, hybridization to the splint (e.g., first splint)brings the terminal nucleotides of the immobilized polynucleotide and the barcoded polynucleotide molecules immediately next to each other, and the ligation does not require gap-filling. In some embodiments, hybridization to first splint (e.g., first splint) brings the terminal nucleotides of the immobilized polynucleotide and the barcoded polynucleotide next to each other and separated by one or more nucleotides, and the ligation is preceded by gap-filling. In some embodiments, the splint is removed after the ligation.

In some embodiments, the method comprises contacting the splint with the lawn of immobilized polynucleotides. Multiple molecules of the splint are flown over the lawn of immobilized polynucleotides. The splint comprises a nucleotide sequence (e.g., first nucleotide sequence) that is complementary to a nucleotide sequence on an polynucleotide (e.g., immobilized polynucleotide). The splint further comprises a nucleotide sequence (e.g., second nucleotide sequence) that is complementary to a nucleotide sequence on a different polynucleotide (e.g., barcoded polynucleotide). Thus, the immobilized polynucleotide is hybridized to a splint which is in turn hybridized to the barcoded polynucleotide in the unmasked region.

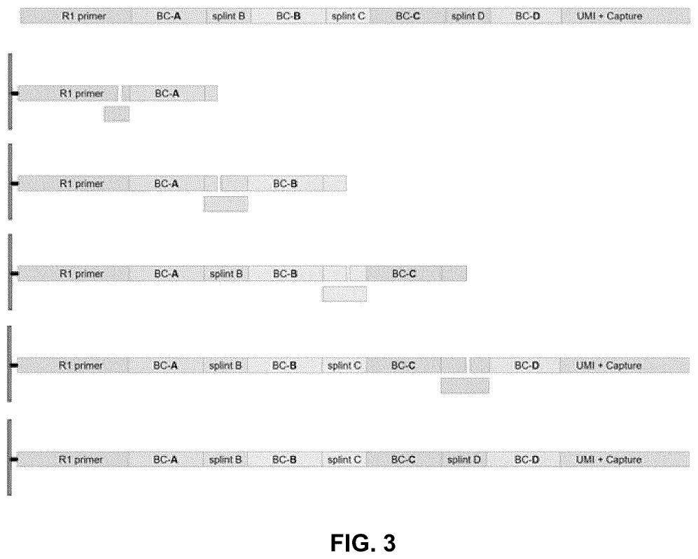

In some aspects, provided herein is a method for construction of a hybridization complex or an array comprising nucleic acid molecules and complexes. FIG. 3 shows an exemplary polynucleotide probe for capturing analytes or proxies thereof which may be generated using a method disclosed herein comprising four rounds of hybridization and ligation.

### B. Copper Catalyzed Click Chemistry-Based DNA Ligation

Disclosed herein is a method for providing an array, comprising: (a) contacting a lawn of polynucleotides immobilized on a substrate with a first barcode sequence, wherein the polynucleotides comprises a 3′-alkyne or 3′-azido group, and wherein the first barcode sequence comprises an polynucleotide with a 5′-azido or 5′-alkyne group that is a reaction partner for a Click reaction with the 3′-alkyne or 3′-azido group of the immobilized polynucleotides; b) masking a first region of the immobilized polynucleotides on the substrate such that only a second region of the immobilized polynucleotides on the substrate are capable of being exposed to light; (c) irradiating the second region of immobilized polynucleotides on the substrate with a first ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the second region with the 5′-azido or 5′-alkyne groups of the first barcode sequence to generate a Click product comprising a 1,2,3-triazole group, thereby providing on the substrate an array comprising the first and second regions of the immobilized polynucleotides, wherein the second region of immobilized polynucleotides is barcoded with the first barcode sequence and the first region of immobilized polynucleotides is not barcoded with the first barcode sequence.

In any of the embodiments herein, the immobilized polynucleotides comprise a 3′-alkyne group and a barcoded polynucleotide (e.g., first barcode sequence) comprises a 5′-azido group. In some embodiments, the immobilized polynucleotides comprise a 3′-azido group and the first barcode sequence comprises a 5′-alkyne group. In any of the embodiments herein, an azide-alkyne click chemistry reaction is performed between the immobilized oligonucleotide and the barcoded oligonucleotide (e.g., first barcode sequence) for ligation.

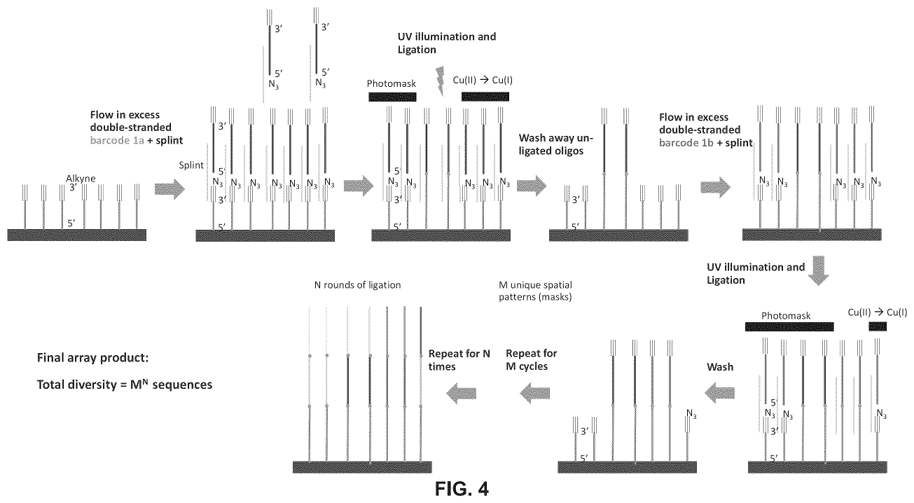

In some embodiments, the immobilized polynucleotides comprise a 3′-alkyne group. In some such embodiments, the first barcode sequence comprises a 5′-azido group that reacts with the 3′-alkyne group of the immobilized polynucleotides, thereby forming a Click reaction product. In some such embodiments, the first barcode sequence also comprises a 3′-alkyne group that is available for a Click reaction with a 5′-azido group on other barcodes (e.g., second and third barcodes) in subsequent rounds of building the array. See FIG. 4.

In some embodiments, the immobilized polynucleotides comprise a 3′-azido group. In some such embodiments, the first barcode sequence comprises a 5′-alkyne group that reacts with the 3′-azido group of the immobilized polynucleotides, thereby forming a Click reaction product. In some such embodiments, the first barcode sequence also comprises a 3′-azido group that is available for a Click reaction with a 5′-alkyne group on other barcodes (e.g., second and third barcodes) in subsequent rounds of building the array.

In any of the embodiments herein, the click chemistry reaction is dependent on a copper compound (e.g., Cu (II)). In some instances, the Cu (II) compound is copper sulfate. The methods disclosed herein comprise irradiating a region of immobilized oligonucleotides or polynucleotides on the substrate with a first ultraviolet light in the presence of a Cu (II) compound and a photoinitiator. Spatially controlled irradiation using ultraviolet light (e.g., 365 nm) facilitates the chemical reduction of Cu (II) to Cu (I). In some aspects, the method is performed in the presence of a photoinitiator molecule. In any of the embodiments herein, the Cu (II) compound and the photoinitiator are flowed over the immobilized lawn of polynucleotides in a solution. A photoinitiator (e.g. Irgacure 2959) creates reactive species (e.g., free radicals, cations or anions) when exposed to UV light. The photoinitiator generate radicals, which reduce Cu (II) to Cu (I). The Cu (I) catalyzes the 1,3-dipolar cycloaddition reaction. The generated Cu(I) catalyzes the Click reaction between the 3′-alkyne or 3′-azido groups of the immobilized polynucleotides in the region with the 5′-azido or 5′-alkyne groups of the barcoded polynucleotide (e.g., first barcode sequence) to generate a Click product comprising a 1,2,3-triazole group. A triazole 5′-azide and 3′-alkyne analog of natural phosphodiester DNA backbone can be formed by Copper-based click chemistry. The Cu(I) is ultimately either reduced to copper metal or dispropotionates to form Cu(0) and Cu(II). An exemplary copper-catalyzed alkyne-azide (CuAAC) click chemistry reaction between is shown below:

### CuAAC Reaction

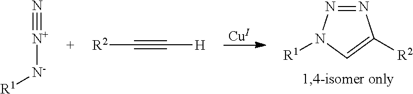

In some embodiments, the immobilized polynucleotides are ligated to the barcoded polynucleotide (e.g., first barcode sequence) using a splint as template. The splint is then removed after ligation.

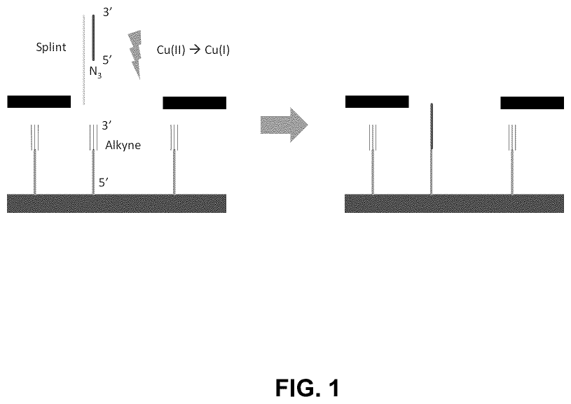

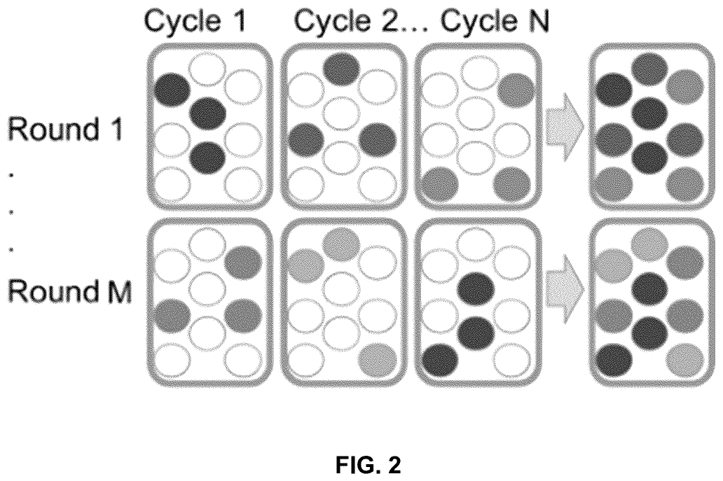

FIG. 1 shows an in situ method for generating a surface array of immobilized polynucleotides, as described herein. A lawn of polynucleotides is immobilized on a substrate. The substrate may be, for example, a glass slide. The lawn of polynucleotides comprise 3′-alkyne groups. A first region of the immobilized polynucleotide on the substrate is masked by using a patterned mask, as shown in FIG. 1. The mask is placed in such a manner that the first region is not capable of being exposed to light, while a second region on the substrate is capable of being exposed to light. Subsequently, a barcode sequence (e.g., first barcode sequence) hybridized to a splint is contacted to the immobilized polynucleotides. The splint comprises nucleotide sequences complementary to the barcode sequence and the immobilized polynucleotide. The barcode sequence comprises a 5′-azido group that is a reaction partner for a click reaction with the 3′-alkyne group on the immobilized polynucleotide in the second exposed region. Next, the substrate is irradiated such that the unmasked region is exposed to the ultraviolet light. Ligation is performed in the presence of a Cu (II) compound and a photoinitiator (e.g. Irgacure 2959). The photoinitiator generate radicals, which reduce Cu (II) to Cu (I). The Cu(I) then catalyzes a 1,3-dipolar cycloaddition reaction between the 3′-alkyne and 5′-azido groups to generate a product comprising a 1,2,3-triazole group. The resulting click product is a ligated extended polynucleotide comprising a barcode sequence. The click product generated is polymerase friendly. Subsequent sets of barcode sequences (e.g., third barcode sequence and/or fourth barcode sequence) may be added by changing the position of the mask to expose the first region and mask the second region, such that only the first region is capable of being exposed irradiation. The immobilized polynucleotide in the first region can then be contacted with a second barcode sequence hybridized to a splint. Irradiation of the first region in the presence of Cu(II) compound and a photoinitiator, generates Cu(I) which catalyzes the Click reaction between 3′-alkyne group on the immobilized polynucleotide and the 5′-azido group on the second barcode sequence. The resulting click product is an extended polynucleotide comprising the second barcode sequence in the first region of the substrate. Using a series of photomasks, polynucleotides in desired regions of the lawn may be iteratively masked. As shown in FIG. 2, the method disclosed herein comprises M rounds, where each round comprises N cycles to achieve a desired barcode diversity up to N^{M}, wherein M and N are integers independent of each other and are at least 2.

Cycles of hybridization and ligation can further add a UMI and a poly(T) domain. Poly(T) sequences are configured to interact with polyadenylated species, such as messenger RNA (mRNA) molecules via the poly(A) tail of an mRNA transcript. Ultimately, poly(T) sequence probes may produce a signal indicating the successful generation of a barcoded polynucleotide array having features approximately 3 microns in diameter.

In some embodiments, the irradiation dose is tuned by selection of the optical setup, including UV lamp power and the duration of irradiation. For example, the choice of a mask aligner which provides focused illumination under an objective lens would provide much greater UV intensity than a un-focused simple benchtop lamp. In some embodiments, the irradiation dose can be about 10, about 15, about 20, about 25, about 30, about 35, about 40, about 45, about 50, about 55, about 60, about 65, about 70, about 75, about 80, about 85, about 90, about 95, or about 100 mW/cm². In some embodiments, the irradiation dose is between about 50 mW/cm² and about 100 mW/cm². For example, the array can be spatially irradiated using 10 mW/cm² for 50 minutes using a 365 nm bandgap filter and a 4000 Acticure lamp.

In some embodiments, the method further comprises contacting the lawn of polynucleotides with a free radical scavenger. The free radical scavenger scavenges the generated Cu(I) ions and prevents non-specific damage of nucleic acids. In some embodiments, the free radical scavenger is dimethyl sulfoxide (DMSO). In some embodiments, the solution comprises DMSO. Any suitable free radical scavengers may be used.

In some embodiments, the method further comprises capping the unreacted 3′ groups in a region (e.g., first and/or second region) of the immobilized polynucleotides. A cycle of hybridization and ligation is followed by a capping reaction to remove unreacted 3′ groups. Single DNA polynucleotides may not all completely undergo successive ligation reactions, leaving available alkyne groups on the surface that may nonspecifically react with the barcoded polynucleotides in the proceeding rounds. To prevent such incomplete reactions a capping step at the end of each round of ligation is performed to remove the unreacted available alkyne groups. In some aspects, the capping step is performed using a monovalent PEG-azide compound. Any suitable compounds may be used for capping the unreacted 3′ groups.

In some aspects, provided herein is a method for construction of a hybridization complex or an array comprising nucleic acid molecules and complexes. FIG. 3 shows an exemplary polynucleotide probe for capturing analytes or proxies thereof which may be generated using a method disclosed herein comprising four rounds of hybridization and ligation.

In some embodiments, the polynucleotide probe for capturing analytes or proxies thereof may be generated from an existing array with a ligation strategy. In some embodiments, an array containing a plurality of polynucleotides (e.g., in situ synthesized oligonucleotides) can be modified to generate a variety of polynucleotide probes. The polynucleotides can include various domains such as, spatial barcodes, UMIs, functional domains (e.g., sequencing handle), cleavage domains, and/or ligation handles.

A “spatial barcode” may comprise a contiguous nucleic acid segment or two or more non-contiguous nucleic acid segments that function as a label or identifier that conveys or is capable of conveying spatial information. In some embodiments, a capture probe includes a spatial barcode that possesses a spatial aspect, where the barcode is associated with a particular location within an array or a particular location on a substrate. A spatial barcode can be part of a capture probe on an array generated herein. A spatial barcode can also be a tag attached to an analyte (e.g., a nucleic acid molecule) or a combination of a tag in addition to an endogenous characteristic of the analyte (e.g., size of the analyte or end sequence(s)). A spatial barcode can be unique. In some embodiments where the spatial barcode is unique, the spatial barcode functions both as a spatial barcode and as a unique molecular identifier (UMI), associated with one particular capture probe. Spatial barcodes can have a variety of different formats. For example, spatial barcodes can include polynucleotide spatial barcodes; random nucleic acid and/or amino acid sequences; and synthetic nucleic acid and/or amino acid sequences. In some embodiments, a spatial barcode is attached to an analyte in a reversible or irreversible manner. In some embodiments, a spatial barcode is added to, for example, a fragment of a DNA or RNA sample before, during, and/or after sequencing of the sample. In some embodiments, a spatial barcode allows for identification and/or quantification of individual sequencing-reads. In some embodiments, a spatial barcode is a used as a fluorescent barcode for which fluorescently labeled oligonucleotide probes hybridize to the spatial barcode.

In some embodiments, a spatial array is generated after ligating capture domains (e.g., poly(T) or gene specific capture domains) to the polynucleotides (e.g., generating capture oligonucleotides). The spatial array can be used with any of the spatial analysis methods described herein. For example, a biological sample can be provided to the generated spatial array. In some embodiments, the biological sample is permeabilized. In some embodiments, the biological sample is permeabilized under conditions sufficient to allow one or more analytes present in the biological sample to interact with the capture probes of the spatial array. After capture of analytes from the biological sample, the analytes can be analyzed (e.g., reverse transcribed, amplified, sequenced) by any of the variety of methods described herein.

For example, FIG. 3 shows the sequential hybridization/ligation of various domains to generate an polynucleotide probe for capturing analytes or proxies thereof, by a photo-hybridization/ligation method described herein.

As illustrated in FIG. 3, an polynucleotide is immobilized on a substrate (e.g., an array) and may comprise a functional sequence such as a primer sequence. In some embodiments, the primer sequence is a sequencing handle that comprises a primer binding site for subsequent processing. The primer sequence can generally be selected for compatibility with any of a variety of different sequencing systems, e.g., 454 Sequencing, Ion Torrent Proton or PGM, Illumina X10, PacBio, Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Roche 454 sequencing, Ion Torrent Proton or PGM sequencing, Illumina X10 sequencing, PacBio SMRT sequencing, and Oxford Nanopore sequencing. Further, in some embodiments, functional sequences can be selected for compatibility with other sequencing systems, including non-commercialized sequencing systems.

Following a first cycle of hybridization and ligation (e.g., copper-catalyzed click chemistry reaction), a barcoded polynucleotide comprising a part of a barcode (e.g., BC-A) is attached to the polynucleotide molecule comprising the primer (e.g., immobilized oligonucleotide or polynucleotide). In some embodiments, the barcode part can be common to all of the polynucleotide molecules in a given feature. In some embodiments, the barcode part can be different for polynucleotide molecules in different features. In some embodiments, a splint with a sequence complementary to a portion of the primer of the immobilized polynucleotide and an additional sequence complementary to a portion of the polynucleotide comprising BC-A (e.g., barcoded polynucleotide) facilitates the ligation of the immobilized polynucleotide and the polynucleotide comprising BC-A (e.g., barcoded polynucleotide). In some embodiments, the splint for attaching part BC-A of various sequences to different features is common among the cycles of the same round. In some embodiments, the ligation of the immobilized polynucleotide and the polynucleotide comprising BC-A is facilitated via a copper-catalyzed click chemistry reaction as described herein. The immobilized polynucleotide comprising the primer further comprises a 3′-alkyne or 3′-azido group and the polynucleotide comprising BC-A further comprises a 5′-azido or 5′-alkyne group. In some aspects, spatially controlled irradiation with a UV light in the presence of a Cu (II) compound and a photoinitiator, facilitates the ligation of the two polynucleotides (e.g., immobilized polynucleotide comprising the primer and the polynucleotide comprising BC-A).

In FIG. 3, another cycle, such as a second cycle, of hybridization and photoligation involves the addition of another polynucleotide comprising a part of a barcode (e.g., BC-B) to the immobilized polynucleotide molecule comprising the primer and BC-A. In some embodiments, a splint with a sequence complementary to a portion of the immobilized polynucleotide comprising BC-A and an additional sequence complementary to a portion of the polynucleotide comprising BC-B facilitates the ligation of the polynucleotide comprising BC-B and the immobilized polynucleotide comprising BC-A. In some embodiments, the splint for attaching part BC-B of various sequences to different features is common among the cycles of the same round. In some embodiments, the ligation of the polynucleotides is facilitated via a copper-catalyzed click chemistry reaction as described herein. In some embodiments, the polynucleotide comprising a part of a barcode (e.g., BC-B) comprises a 5′-azido or 5′-alkyne group. In some embodiments, the immobilized polynucleotide comprising BC-A comprises a 3′-alkyne or 3′-azido group. In some aspects, spatially controlled irradiation with a UV light in the presence of a Cu (II) compound and a photoinitiator facilitates the ligation of the two polynucleotides (e.g., the immobilized polynucleotide comprising BC-A, and the polynucleotide comprising BC-B).

FIG. 3 further illustrates a third cycle of hybridization and photoligation, which involves the addition of another polynucleotide comprising a part of a barcode (e.g., BC-C), added to the immobilized polynucleotide molecule comprising the primer, BC-A, and BC-B. In some embodiments, a splint with a sequence complementary to a portion of the immobilized polynucleotide molecule comprising BC-B and an additional sequence complementary to a portion of the polynucleotide comprising BC-C facilitates the ligation of the immobilized polynucleotide molecule comprising BC-B and the polynucleotide comprising BC-C. In some embodiments, the splint for attaching part BC-C of various sequences to different features is common among the cycles of the same round. In some embodiments, the ligation of the polynucleotides is facilitated via a copper-catalyzed click chemistry reaction as described herein. In some embodiments, the polynucleotide comprising a part of a barcode (e.g., BC-C) comprises a 5′-azido or 5′-alkyne group. In some embodiments, the immobilized polynucleotide comprising BC-B comprises a 3′-alkyne or 3′-azido group. In some aspects, spatially controlled irradiation with a UV light in the presence of a Cu (II) compound and a photoinitiator facilitates the ligation of the two polynucleotides (e.g., the immobilized polynucleotide comprising BC-B, and the polynucleotide comprising BC-C).

A fourth cycle of hybridization and photoligation may be performed, which involves the addition of another polynucleotide comprising a part of a barcode (e.g., BC-D), added to the immobilized polynucleotide molecule comprising the primer, BC-A, BC-B, and BC-C. In some embodiments, a splint with a sequence complementary to a portion of the immobilized polynucleotide molecule comprising BC-C and an additional sequence complementary to a portion of the polynucleotide comprising BC-D facilitates the ligation. In some embodiments, the splint for attaching part BC-D of various sequences to different features is common among the cycles of the same round. In some embodiments, as shown in FIG. 3, an polynucleotide comprising BC-D further comprises a UMI and a capture domain. In some embodiments, the ligation of the polynucleotides is facilitated via a copper-catalyzed click chemistry reaction as described herein. In some embodiments, the polynucleotide comprising a part of a barcode (e.g., BC-C) comprises a 5′-azido or 5′-alkyne group. In some embodiments, the immobilized polynucleotide comprising BC-D comprises a 3′-alkyne or 3′-azido group. In some aspects, spatially controlled irradiation with a UV light in the presence of a Cu (II) compound and a photoinitiator facilitates the ligation of the two polynucleotides (e.g., the immobilized polynucleotide comprising BC-C, and the polynucleotide comprising BC-D).

In some embodiments, polynucleotides that are exposed and do not receive a ligated polynucleotide could receive the incorrect barcode during the next cycle or round. In order to prevent generating the wrong barcode at the wrong spot, unligated polynucleotides may be rendered unavailable for hybridization and/or ligation, e.g., the unligated oligonucleotides can be capped and/or removed. In some embodiments, the polynucleotides are modified at the 3′. Non-limiting examples of 3′ modifications include dideoxy C-3′ (3′-ddC), 3′ inverted dT, 3′ C3 spacer, 3′Amino, and 3′ phosphorylation. In some embodiments, capping of unligated oligonucleotides at their 3′ ends is performed using a monovalent PEG-azide.

In some embodiments according to any of the methods described herein, the method further comprises blocking the 3′ or 5′ termini of barcoded polynucleotide molecules. In some embodiments, the method further comprises blocking the unligated polynucleotide molecules in the region (e.g., first and/or second region) from ligation. In some embodiments, the blocking comprises adding a 3′ dideoxy or a non-ligating 3′ phosphoramidite to the barcoded polynucleotide molecules. In some embodiments, the blocking comprises adding a 3′ dideoxy or a non-ligating 3′ phosphoramidite to the barcoded polynucleotide molecules. In some embodiments, the blocking comprises adding a 3′ dideoxy or a non-ligating 3′ phosphoramidite to the unligated polynucleotide molecules. In some embodiments, the addition is catalyzed by a terminal transferase. In some embodiments, the terminal transferase is TdT. The blocking may be removed after the blocking reaction is completed. In some embodiments, the blocking is removed using an internal digestion of the barcoded polynucleotide molecules after ligation is completed.

In some embodiments the method for providing an array comprises: (a) contacting a lawn of polynucleotides immobilized on a substrate with a plurality of first barcode sequences (e.g., first polynucleotides comprising first barcode sequences) and a first splint (FIG. 4). The lawn of polynucleotides comprises a 3′-alkyne or 3′-azido group, and the first barcode comprises an polynucleotide with a 5′-azido or 5′-alkyne group that is a reaction partner for a Click reaction with the 3′-alkyne or 3′-azido group of the immobilized polynucleotides. In some aspects, step (a) comprises masking a second region of the immobilized polynucleotides on the substrate such that a first region of the immobilized polynucleotides is capable of being exposed to light. In some aspects, the immobilized polynucleotides in the second region are protected by the photomask from hybridization and/or ligation (FIG. 4). In some embodiments, the first splint hybridizes to the first barcode sequence (e.g., first polynucleotides comprising first barcode sequences) and the immobilized polynucleotide molecules in the first region. In some embodiments, the method further comprises (b) irradiating the first region of immobilized polynucleotides on the substrate with a first ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the first region with the 5′-azido or 5′-alkyne groups of the first barcode sequence to generate a Click product (e.g., first extended oligonucleotide molecule). Thus, the method provides on the substrate an array comprising the first and second regions of the immobilized polynucleotides, wherein the first region of immobilized polynucleotides is barcoded with the first barcode sequence and the second region of immobilized polynucleotides is not barcoded with the first barcode sequence.

In some embodiments, the method further comprises the following steps: c) contacting the immobilized polynucleotides on the substrate with a plurality of second polynucleotides comprising a second barcode sequence and a second splint. In some embodiments, step c) comprises masking the first region of immobilized polynucleotides on the substrate so that the second region of immobilized polynucleotides is capable of being exposed to light and is available for hybridization and/or ligation. The first extended polynucleotide molecules in the first region are protected by the photomask in the first region from hybridization and/or ligation. In some embodiments, the second splint hybridizes to the second polynucleotide and the polynucleotide molecules in the second region.

In some embodiments, the method further comprises: d) irradiating the second region of immobilized polynucleotides on the substrate with a first ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the second region with the 5′-azido or 5′-alkyne groups of the second barcode sequence of the second polynucleotide to generate a Click product (e.g., second extended polynucleotide molecule). In some embodiments, the second polynucleotide is ligated to the immobilized polynucleotides in the second region to generate second extended polynucleotides. In some embodiments, the second polynucleotide is not ligated to the first extended polynucleotide molecules in the first region.

In some embodiments according to the methods described in the section, steps (a)-(b) are part of a first cycle, steps (c)-(d) are part of a second cycle, and steps (a)-(d) are part of a first round, and wherein the method comprises one or more additional rounds. In some embodiments, steps (a)-(d) are part of a first round, the first and second polynucleotides are Round 1 polynucleotides, the first and second barcode sequences are Round 1 barcode sequences.

In some embodiments, the method further comprises: a′) contacting a plurality of first Round 2 polynucleotides comprising a first Round 2 barcode sequences to the first and second extended polynucleotides in the first and second region. The first and second extended polynucleotides in the first and second region comprise 3′-alkyne or 3′-azido groups, and the plurality of first Round 2 polynucleotides comprises 5′-azido or 5′-alkyne groups that are reaction partners for a Click reaction with the 3′-alkyne or 3′-azido group of the first and second extended polynucleotides. In some aspects, step (a′) comprises masking the second region of the second extended polynucleotides on the substrate such that the first extend polynucleotides in the first region are capable of being exposed to light. In some aspects, the second extended polynucleotides in the second region are protected by the photomask from hybridization and/or ligation. In some embodiments, the splint hybridizes to the first extended polynucleotides and the Round 2 polynucleotides comprising the first Round 2 barcode sequences. In some embodiments, the method further comprises (b′) irradiating the first region of first extended polynucleotides on the substrate with a first ultraviolet light in the presence of a Cu (II) compound and a photoinitiator, thereby generating Cu(I), wherein the Cu(I) catalyzes the Click reaction between the 3′-alkyne or 3′-azido groups of the first extended polynucleotides in the first region with the 5′-azido or 5′-alkyne groups of the Round 2 polynucleotides to generate a Click product (e.g., a first further extended polynucleotide) comprising the first Round 2 polynucleotides. The second extended polynucleotide molecules in the second region are protected from ligation by the photomask and are not ligated to the first Round 2 barcode sequence.

In some embodiments, the method further comprises the following steps: (c′) contacting a plurality of second Round 2 polynucleotides comprising a second Round 2 barcode sequences to the first further extended polynucleotides and the second extended polynucleotides in the first and second region. In some embodiments, step (c′) comprises masking the first region of first further extended polynucleotides so that the second region of second extended polynucleotides is capable of being exposed to light and is available for hybridization and/or ligation. The first further extended polynucleotide molecules in the first region are protected by the photomask in the first region from hybridization and/or ligation. In some embodiments, the splint hybridizes to the second extended polynucleotides and the second Round 2 polynucleotides comprising a second Round 2 barcode sequences in the second region.

In some embodiments, the method further comprises: (d′) irradiating the second region of second extended polynucleotides with a first ultraviolet light in the presence of a Cu (II) compound and a photoinitiator, thereby generating Cu(I), wherein the Cu(I) catalyzes the Click reaction between the 3′-alkyne or 3′-azido groups of second extended polynucleotides in the second region with the 5′-azido or 5′-alkyne groups of the second Round 2 polynucleotides to generate a Click product (e.g., second further extended polynucleotide) comprising the second Round 2 polynucleotides. In some embodiments, the second Round 2 polynucleotides are ligated to the second extended polynucleotides in the second region to generate second further extended polynucleotide. In some embodiments, the second Round 2 polynucleotides are not ligated to the first further extended polynucleotide in the first region.

In some embodiments, the Round 1 barcode sequences are different from each other. In some embodiments, the Round 1 barcode sequences are different from the Round 2 barcode sequences. In some embodiments, the method is repeated for M cycles and N rounds to generate a total barcode diversity of M^{N} sequences.

In some embodiments, the method for providing an array comprises a photoresist. In some embodiments the method for providing an array comprises: (a) irradiating a substrate comprising an unmasked first region and a masked second region, whereby a photoresist in the first region is degraded to render immobilized r polynucleotide molecules in the first region available for hybridization and/or ligation, whereas immobilized polynucleotide molecules in the second region are protected by a photoresist from hybridization and/or ligation. In some embodiments according to the method for providing an array described herein, the photoresist is a first photoresist. In some embodiments, the method further comprises (b) contacting the immobilized polynucleotide molecules in the first region with a first barcode sequence and irradiating the region with a first ultraviolet light in the presence of a Cu (II) compound and a photoinitiator. The Cu(I) catalyzes the Click reaction between the 3′-alkyne or 3′-azido groups of the immobilized polynucleotides in the first region with the 5′-azido or 5′-alkyne groups of the first barcode sequence to generate a Click product (e.g., first extended polynucleotide molecules). In some embodiments, the immobilized polynucleotide in the first region is ligated to the first barcode sequence in the first region via copper-catalyzed alkyne-azide click chemistry to generate first extended polynucleotide molecule.

In some embodiments, the method further comprises the following steps: c) applying a second photoresist to the substrate, optionally wherein the second photoresist is applied after the first photoresist is removed from the substrate. In some embodiments, the method further comprises d) irradiating the substrate while the first region is masked and the second region is unmasked, whereby the second photoresist in the second region is degraded to render polynucleotide molecules in the second region available for hybridization and/or ligation, whereas the first extended polynucleotide molecules in the first region are protected by the second photoresist in the first region from hybridization and/or ligation. In some embodiments, the method further comprises e) contacting polynucleotide molecules in the second region with a second splint and a second polynucleotide comprising a second barcode sequence and irradiating the second region in the presence of a Cu (II) compound and a photoinitiator. In some embodiments, the second splint hybridizes to the second polynucleotide and the polynucleotide molecules in the second region. The Cu(I) catalyzes the Click reaction between the 3′-alkyne or 3′-azido groups of the polynucleotide molecules in the second region with the 5′-azido or 5′-alkyne groups of the second polynucleotide to generate a Click product (e.g., second extended polynucleotide molecules). In some embodiments, the second polynucleotide is ligated to the polynucleotide molecules in the second region to generate second extended polynucleotide molecules. In some embodiments, the second polynucleotide is not ligated to the first extended polynucleotide molecules in the first region.

In some embodiments according to the methods described in the section, steps (a)-(b) are part of a first cycle, steps (d)-(e) are part of a second cycle, and steps (a)-(e) are part of a first round, and wherein the method comprises one or more additional rounds. In some embodiments, steps (a)-(e) are part of a first round, the first and second polynucleotides are Round 1 polynucleotides, the first and second barcode sequences are Round 1 barcode sequences. In some embodiments, the method further comprises: a′) irradiating the substrate while the first region is unmasked and the second region is masked, whereby a photoresist in the first region is degraded to render the first extended polynucleotide molecules in the first region available for hybridization and/or ligation, whereas the second extended polynucleotide molecules in the second region are protected by the photoresist in the second region from hybridization and/or ligation; and (b′) attaching a first Round 2 polynucleotide comprising a first Round 2 barcode sequence to the first extended polynucleotide molecules in the first region via hybridization and copper-catalyzed azide alkyne click chemistry ligation, wherein the second extended polynucleotide molecules in the second region do not receive the first Round 2 barcode sequence. In some embodiments wherein the photoresist is a first photoresist, and the first Round 2 polynucleotide is ligated to the first extended polynucleotide molecules in the first region to generate first further extended polynucleotide molecules, the method further comprises: (c′) applying a second photoresist to the substrate, optionally wherein the second photoresist is applied after the first photoresist is removed from the substrate; (d′) irradiating the substrate while the first region is masked and the second region is unmasked, whereby the first or second photoresist in the second region is degraded to render the second extended polynucleotide molecules in the second region available for hybridization and/or ligation, whereas the first further extended polynucleotide molecules in the first region are protected by the second photoresist in the first region from hybridization and/or ligation; and (e′) attaching a second Round 2 polynucleotide comprising a second Round 2 barcode sequence to the second extended polynucleotide molecules in the second region via hybridization and copper-catalyzed azide alkyne click chemistry ligation, wherein the first further extended polynucleotide molecules in the first region do not receive the second Round 2 barcode sequence. In some embodiments, the Round 1 barcode sequences are different from each other. In some embodiments, the Round 1 barcode sequences are different from the Round 2 barcode sequences.

### IV. Light-Controlled Surface Patterning In Situ Using Click Chemistry

Provided herein is a method of patterning a surface in situ for producing an array on the surface, for example, by spatially-selective light-activated hybridization and ligation generating unique DNA sequences in unique spatial positions in the array. In some embodiments, the method comprises assembling barcode sequences on immobilized polynucleotides, e.g., based on hybridization and copper-catalyzed click chemistry based ligation, on a slide or wafer surface. In some embodiments, the method avoids multiple steps of photo-activated deprotection and enzymatic ligation steps. The method allows for direct ligation of the polynucleotides using a one-step photo activated click chemistry reaction. In some embodiments, the click chemistry reaction is dependent on a copper compound (e.g., Cu (II)). In some embodiments, irradiation with ultraviolet light in the presence of a Cu (II) compound and a photoinitiator generates Cu(I), wherein the Cu(I) catalyzes the Click reaction between the 3′-alkyne or 3′-azido groups and the 5′-azido or 5′-alkyne groups of the polynucleotides (e.g., barcode sequences) to generate a Click product comprising a 1,2,3-triazole group.

In some embodiments, the in situ method comprises photolithography, and optionally using one or more photoresist compositions to enable barcodes to be generated combinatorially, for example, in as few as three rounds of assembly. The method may not use a photoresist. In some embodiments, exposure of the photoresist to irradiation may render the exposed regions dissolvable by a developer. In some embodiments, the photoresist in the unmasked region of the substrate is dissolved by a developer and removed. The developer may be organic or aqueous based. A non-limiting example of an aqueous base developer such as tetramethylammonium hydroxide aqueous solution.

In some aspects, provided herein is a method of patterning a surface in situ for producing an array on the surface. In some embodiments, the method comprises assembling barcode sequences on immobilized polynucleotides, e.g., based on hybridization and click chemistry based ligation, on a slide surface. In some embodiments, the in situ method uses photolithography and click chemistry reactions to enable barcodes to be generated selectively on a discrete location on a slide surface. Hybridization and click chemistry based ligation of barcodes can be spatially controlled, for example, using a contact photolithography process. For example, ligation can be achieved by using a photomask to expose oligonucleotides and irradiating the unmasked region for Cu(I) catalyzed ligation.

In some embodiments, the polynucleotide molecules on the substrate comprise one or more common sequences. In some embodiments, the one or more common sequences comprise a common primer sequence. The common primer sequence can be of about 5, about 6, about 7, about 8, about 9, about 10, about 15, about 20, about 25, about 30, about 35, about 40, about 45, about 50, or about 60 nucleotides in length. In some embodiments, the common primer sequence is between about 10 and about 35 nucleotides in length.

In some embodiments, the polynucleotide molecules in the first region and polynucleotide molecules in the second region are identical in sequence. In some embodiments, the polynucleotide molecules on the substrate prior to the irradiating step are identical in sequence.

In some embodiments, polynucleotide molecules in the first and the second regions are different. In some embodiments, polynucleotide molecules in the first region and polynucleotide molecules in the second region are different in sequences. In some embodiments, polynucleotide molecules in the first region and polynucleotide molecules in the second region comprise different barcode sequences. In some embodiments, polynucleotide molecules on the substrate comprise two or more different sequences.

In some embodiments, the array comprises an arrangement of a plurality of features, e.g., each comprising one or more molecules such as a nucleic acid molecule (e.g., a DNA polynucleotide). In some embodiments, the array comprises different polynucleotides in different features. In some embodiments, polynucleotide molecules on the substrate are immobilized in a plurality of features. Nucleotides immobilized on the substrate may be of different orientations. For example, in some embodiments, the 3′ terminal nucleotides of immobilized polynucleotide molecules are distal to the substrate. In some embodiments, the 5′ terminal nucleotides of immobilized polynucleotide molecules are distal to the substrate.

The polynucleotide molecules on the substrate prior to the irradiating step may have a variety of properties, which include but are not limited to, length, orientation, structure, and modifications. The polynucleotide molecules on the substrate prior to the irradiating step can be of about 5, about 6, about 7, about 8, about 9, about 10, about 15, about 20, about 25, about 30, about 35, about 40, about 45, about 50, about 55, about 60, about 70, about 80, about 90, or about 100 nucleotides in length. In some embodiments, polynucleotide molecules on the substrate prior to the irradiating step are between about 5 and about 50 nucleotides in length. The polynucleotide molecules on the substrate may comprise functional groups. In some embodiments, the functional groups are 3′-alkyne or 3′-azido groups. In some embodiments, the functional groups are 5′-alkyne or 5′-azido groups. The functional groups can be protected or unprotected. In some embodiments, the functional groups are not protected, e.g., by a photo-sensitive group, moiety, or molecule prior to the irradiating step.

In some embodiments, the method provided herein comprises forming a pattern of polynucleotide molecules on the substrate prior to applying the photoresist to the substrate. For example, the pattern of polynucleotide can be formed by irradiating a substrate comprising a plurality of functional groups and a photoresist through a patterned mask, whereby the photoresist in a first region of the substrate is degraded, rendering functional groups in the first region available for reacting with functional groups in functionalized polynucleotide molecules, whereas functional groups in a second region of the substrate are protected by the photoresist from reacting with functional groups in the polynucleotide molecules; and contacting the substrate with the functionalized polynucleotide molecules, wherein the functionalized polynucleotide molecules are coupled to functional groups in the first region but not to functional groups in the second region.

In some embodiments, the plurality of functional groups of the substrate are not protected, e.g., by a photo-sensitive group, moiety, or molecule prior to the irradiating step.

In some embodiments, the method further comprises heating the substrate to dryness during or after the contacting step. In some embodiments, the method further comprises blocking unreacted functional groups of the substrate. In some embodiments, the method further comprises rendering the reaction between functional groups of the substrate and the functionalized polynucleotide molecules irreversible.

In some embodiments, provided herein is a method to generate an array with barcode diversity in the 100s, 1,000s, 10,000s, 100,000s, 1,000,000s, or 10,000,000s. In some embodiments, a substrate comprising a dense lawn of a common polynucleotide is provided and may be protected with a photoresist layer. Using a series of photomasks, polynucleotides in desired regions of the lawn may be iteratively masked using a photomask. In some embodiments, the method further comprises performing a click chemistry reaction described herein and attaching a round 1 barcode to one or more exposed polynucleotides, for example, by attaching an polynucleotide cassette with a complementary region (e.g., complementary to a splint) and a barcode region. In some embodiments, the attachment may be performed by placing the substrate in a chamber or vessel (e.g., within which oligonucleotides such as those comprising barcode sequences can be delivered and ligated to nucleic acid molecules on the substrate). In some embodiments, the chamber or vessel is a flow cell or a device comprising microfluidic channels. In some embodiments, the method comprises flowing in the round 1 barcode (e.g., an polynucleotide cassette) to be attached to the common polynucleotide. The process can be repeated N cycles (each cycle for one or more features on an array) for round 1 until all desired features have been exposed (e.g., due to exposure of the photoresist covering the features to light) and the common polynucleotides in the features have received the round 1 barcode which may be the same or different for molecules in any two given features. The round 1 barcode molecules can be ligated via a click chemistry reaction as described herein to the common polynucleotides. The process can be repeated M rounds to achieve a desired barcode diversity, for example, by attaching a round 2 barcode (which may be the same or different for molecules in any two given features), a round 3 barcode (which may be the same or different for molecules in any two given features), . . . , and a round m barcode (which may be the same or different for molecules in any two given features) to each of the growing polynucleotides in the features. In some embodiments, each round comprises a plurality of cycles (each cycle for one or more features on an array) of irradiation and polynucleotide attachment and/or photoresist degradation until all desired features have been exposed once and the molecules in the features have received the barcode(s) (which may be the same or different for molecules in any two given features) for that round. In some embodiments, the method further comprises attaching a capture sequence to the barcoded polynucleotides, for example, by hybridization and click chemistry based ligation.

In some aspects, a method disclosed herein provides one or more advantages as compared to other arraying methods. For example, pre-synthesized barcodes can eliminate concerns over barcode fidelity in base-by-base in situ approaches. In addition, compared to base-by-base methods, the method disclosed herein can reduce time and increase total yield. For example, only three or four rounds of hybridization and ligation may be required compared to 12-16 rounds in a typical base-by-base in situ arraying method. In one aspect, the method disclosed herein does not involve 5′ to 3′ base-by-base synthesis of a polynucleotide in situ on a substrate. In another aspect, there is no need for decoding the arrays, as all barcodes are synthesized in defined locations on an array. In some aspects, feature scaling can readily be increased or decreased by changing photomasks and corresponding barcode diversity. In other aspects, a method disclosed herein is performed on a transparent substrate. Since a method disclosed herein does not depend on the use of microspheres (e.g., barcoded beads) to generate an oligonucleotide array, optical distortion or aberrations caused by microspheres (which may not be transparent) during imaging of the polynucleotide array and/or a sample (e.g., a tissue section) on the array can be avoided.

In some aspects, provided herein is a method of producing an array of polynucleotides. In some embodiments, an array comprises an arrangement of a plurality of features, e.g., each comprising one or more molecules such as a nucleic acid molecule (e.g., a DNA oligonucleotide), and the arrangement is either irregular or forms a regular pattern. The features and/or molecules on an array may be distributed randomly or in an ordered fashion, e.g. in spots that are arranged in rows and columns. Individual features in the array differ from one another based on their relative spatial locations. In some embodiments, the features and/or molecules are collectively positioned on a substrate.

In some embodiments, polynucleotides of the same or different nucleic acid sequences are immobilized on the substrate in a pattern prior to the irradiation. In some embodiments, the pattern comprises rows and/or columns. In some embodiments, the pattern comprises regular and/or irregular shapes (e.g., polygons).

In some embodiments, the method comprises irradiating an array with light. In some embodiments, the irradiation is selective, for example, where one or more photomasks can be used such that only one or more specific regions of the array are exposed to stimuli (e.g., exposure to light such as UV, and/or exposure to heat induced by laser). In some embodiments, the method comprises irradiating a first region of a substrate with a first light while a second region of the substrate is not irradiated with the first light. For instance, the substrate is exposed to the first light when the second region is photomasked while the first region is not photomasked. Alternatively, a focused light such as laser may be used to irradiate the first region but not the second region, even when the second region is not masked from the light. For example, the distance (pitch) between features may be selected to prevent the laser from degrading photoresist protecting polynucleotides of an adjacent feature.

In some embodiments, the photoresist inhibits or blocks hybridization and/or ligation of polynucleotide molecules in the first region and/or the second region. In some embodiments, the polynucleotide molecules are prevented by the photoresist from hybridization to a nucleic acid such as a splint. In some embodiments, the polynucleotide molecules are prevented by the photoresist from ligation to a nucleic acid. For example, the photoresist may inhibit or block the 3′ or 5′ end of an polynucleotide molecule from chemical or enzymatic ligation, e.g., even when a splint may hybridize to the polynucleotide molecule in order to bring a ligation partner in proximity to the 3′ or 5′ end of the polynucleotide molecule. In some embodiments, the 3′ or 5′ end of the polynucleotide molecule or a hybridization/ligation product thereof is capped.

In some embodiments, the irradiation results in ligation to an polynucleotide molecule in an exposed (e.g., unmasked) region, whereas ligation to an polynucleotide molecule in an unexposed (e.g., masked) region remains inhibited or blocked by a photomask.

In some embodiments, a photoresist may be used to generate an array. In some embodiments, the irradiation results in degradation of the photoresist such that the inhibition or blocking of hybridization and/or ligation to an polynucleotide molecule in an exposed (e.g., unmasked) region is reduced or eliminated, whereas hybridization and/or ligation to an polynucleotide molecule in an unexposed (e.g., masked) region remains inhibited or blocked by a photoresist which may be the same or different from the degraded photoresist.

In some embodiments, the method further comprises attaching a first barcode molecule comprising a first barcode sequence to an polynucleotide molecule in an exposed (e.g., unmasked) region via hybridization and ligation. In some embodiments, one end of the first barcode molecule and one end of the polynucleotide molecule may be directly ligated, e.g., using copper catalyzed click chemistry. The attachment may comprise hybridizing the first barcode molecule and the polynucleotide molecule to a splint, wherein one end of the first barcode molecule and one end of the polynucleotide molecule are in proximity to each other. For example, the 3′ end of the first barcode molecule and the 5′ end of the polynucleotide molecule may hybridize to a splint. Alternatively, the 5′ end of the first barcode molecule and the 3′ end of the polynucleotide molecule are in proximity to each other.

In any of the preceding embodiments, physical masks, e.g., a photolithography mask which is an opaque plate or film with transparent areas that allow light to shine through in a defined pattern, may be used.

In some embodiments, a first polynucleotide (e.g., an oligonucleotide) is deposited in a region A of a substrate and a second polynucleotide (e.g., an oligonucleotide) is deposited in a region B. Regions A are exposed to light while regions B are masked by a photomask. A photomask can be selected and/or adjusted to allow any suitable number and/or combination of regions on the substrate to be exposed to light or masked. Thus, the exposed region(s) and masked region(s) can be in any suitable pattern, which can be predetermined and/or adjusted as needed during the arraying process. In addition, a mirror, mirror array, a lens, a moving stage, and/or a photomask can be used to direct the light to or away from the region(s) of interest. In some embodiments, the first polynucleotide and the second polynucleotide can comprise the same sequence or different sequences. For example, first polynucleotides in region A and second polynucleotides in region B may form a lawn of universal polynucleotide molecules on the substrate. The polynucleotides may be attached to the substrate at their 5′ ends or 3′ ends. The first and second polynucleotides can be embedded in a first and a second photoresist, respectively. The first and second photoresist can be the same or different. In some embodiments, first polynucleotides in region A and second polynucleotides in region B are embedded in the same photoresist layer. Once regions A are exposed to light to deprotect the first polynucleotide while the second polynucleotide in regions B remain protected, a first barcode can be attached to the first polynucleotide. In some embodiments, a hybridization complex is formed between the first polynucleotide, a splint, and a polynucleotide comprising a first barcode (e.g., a round 1 barcode 1A). The polynucleotide comprising the first barcode comprise at least a first barcode sequence and a hybridization region that hybridizes to the splint which is a first splint, and may further comprise a hybridization region that hybridizes to a round 2 splint (e.g., for attaching a round 2 barcode after the round 1 barcode 1A). The first splint comprises at least a hybridization region that hybridizes to the first polynucleotide and a hybridization region that hybridizes to the polynucleotide comprising the first barcode. Optionally, the polynucleotide comprising the first barcode may be ligated to the first polynucleotide, without gap filling using the first splint as a template. As a result, provided in some embodiments is an array comprising the first and second polynucleotides, wherein the first polynucleotide is barcoded with the first barcode and the second polynucleotide is not, and neither of the barcoded first polynucleotide nor the second polynucleotide comprises a photo-cleavable moiety.

In some embodiments, the polynucleotide comprising the first barcode may comprise no photo-cleavable moiety that blocks hybridization and/or ligation. In these examples, the array may be exposed to light to degrade photoresist that protects the second polynucleotide, and a second barcode can be attached to the second polynucleotide. In some embodiments, a hybridization complex is formed between the second polynucleotide, a second splint, and a polynucleotide comprising a second barcode (e.g., a round 1 barcode 1B). The polynucleotide comprising the second barcode comprises at least a second barcode sequence and a hybridization region that hybridizes to the second splint, and may further comprise a hybridization region that hybridizes to a round 2 splint (e.g., for attaching a round 2 barcode after the round 1 barcode 1B). The second splint comprises at least a hybridization region that hybridizes to the second polynucleotide and a hybridization region that hybridizes to the polynucleotide comprising the second barcode. While the polynucleotide comprising the first barcode may be available for hybridization and/or ligation, the second barcode may be specifically attached to the second polynucleotide but not to the first polynucleotide barcoded with the first barcode. For example, the sequence of the second splint may be selected such that it specifically hybridizes to the second polynucleotide but not to the polynucleotide comprising the first barcode. In these examples, both the first barcode (e.g., barcode 1A) and the second barcode (e.g., barcode 1B) are round 1 barcodes. Optionally, the polynucleotides comprising the first/second barcodes may be ligated to the first/second polynucleotides, respectively, without gap filling using the first/second splints as templates. As a result, provided in some embodiments is an array comprising the first and second polynucleotides barcoded with the first barcode and the second barcode, respectively, wherein neither of the barcoded polynucleotides comprises a photo-cleavable moiety.

In some examples, polynucleotides in regions A and/or polynucleotides in regions B may undergo to one or more additional rounds of barcoding. For example, after the round 1 barcoding, regions A may contain polynucleotides P1 and P3 each barcoded with round 1 barcode 1A (i.e., polynucleotides 1A-P1 and 1A-P3) and regions B may contain polynucleotides P2 and P4 each barcoded with round 1 barcode 1B (i.e., polynucleotides 1B-P2 and 1B-P4). All of polynucleotides 1A-P1, 1A-P3, 1B-P2, and 1B-P4 may be embedded in a photoresist. With light exposure and photomasking, any one or more of polynucleotides 1A-P1 and 1A-P3 (in regions A) and 1B-P2 and 1B-P4 (in regions B) may undergo a second round of barcoding.

For instance, a round 2 barcode 2A may be attached to any one of polynucleotides 1A-P1, 1A-P3, 1B-P2, and 1B-P4. In some embodiments, a round 2 barcode 2A may be attached to any two of polynucleotides 1A-P1, 1A-P3, 1B-P2, and 1B-P4. In some embodiments, a round 2 barcode 2A may be attached to any three of polynucleotides 1A-P1, 1A-P3, 1B-P2, and 1B-P4. In some embodiments, a round 2 barcode 2A may be attached to all of polynucleotides 1A-P1, 1A-P3, 1B-P2, and 1B-P4.

In other examples, different round 2 barcodes 2A and 2B may be used. In some embodiments, barcode 2A is attached to polynucleotides 1A-P1 and 1A-P3 (in regions A) while barcode 2B is attached to polynucleotides 1B-P2 and 1B-P4 (in regions B). For higher order rounds, for example, round m (m being an integer of 2 or greater), the regions A polynucleotides may receive barcode mA while the regions B polynucleotides receive barcode mB. Barcodes mA and mB may be the same or different in sequence. Thus, for each round, the regions A polynucleotides (e.g., P1 and P3) and the regions B polynucleotides (e.g., P2 and P4) may have no crossover, generating barcoded polynucleotides mA- . . . -1A-P1 and mA- . . . -1A-P3 (in regions A) and mB- . . . -1B-P2 and mB- . . . -1B-P4 (in regions B).

Alternatively, the regions A polynucleotides (e.g., P1 and P3) and the regions B polynucleotides (e.g., P2 and P4) may have crossover. For example, barcode 2A is attached to polynucleotides 1A-P1 (in regions A) and 1B-P2 (in regions B) while barcode 2B is attached to polynucleotides 1A-P3 (in regions A) and 1B-P4 (in regions B). For example, barcoded polynucleotides 2A-1A-P1 and 2B-1A-P3 (in regions A) ans 2A-1B-P2 ans 2B-1B-P4 (in regions B) may be generated. For round m (m being an integer of 2 or greater), one or more of the regions A polynucleotides and/or one or more of the regions B polynucleotides may receive barcode mA, while one or more of the regions A polynucleotides and/or one or more of the regions B polynucleotides barcode mB. Barcodes mA and mB may be the same or different in sequence.

In some examples, round m (m being an integer of 2 or greater) barcodes mA, mB, and mC may be attached to any polynucleotides barcoded in the previous round (i.e., round m-1), and mA, mB, and mC may be the same or different. In other examples, round m (m being an integer of 2 or greater) barcodes mA, mB, mC, and mD may be attached to any polynucleotides barcoded in the previous round (i.e., round m-1), and mA, mB, mC, and mD may be the same or different.

In any of the preceding embodiments, the barcoding rounds can be repeated m times to achieve a desired barcode diversity, m being an integer of 2 or greater. In some embodiments, m is 3, 4, 5, 6, 7, 8, 9, or 10, or greater than 10. In any of the preceding embodiments, each of the m barcoding rounds may comprise n cycles (each cycle for molecules in one or more features), wherein integer n is 2 or greater and independent of m. In some embodiments, n is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater than 50.

FIG. 4 provides a non-limiting example, where the substrate comprises a surface for nucleic acids to be deposited on and can be in the form of a slide, such as a glass slide. In some examples, the substrate is transparent. A lawn of polynucleotides without photo-cleavable moieties, such as photo-caged oligonucleotides, may be deposited on the substrate and immobilized. A photoresist may be coated onto the substrate and cover the polynucleotides. One or more regions (e.g., regions A) on the substrate are unmasked using a patterned mask, rendering the polynucleotides in the one or more regions available for hybridization and ligation, while one or more other regions (e.g., regions B) on the substrate are masked or not exposed to light. Patterned exposure of polynucleotides on the underlying substrate is provided, and a round 1 barcode (such as barcode 1A) may be attached to the exposed polynucleotides via hybridization and click chemistry based ligation. For example, an oligonucleotide may be used to hybridize to an exposed polynucleotide and a polynucleotide comprising the round 1 barcode. The oligonucleotide may comprise a splint that facilitates proximity ligation of one end of the exposed polynucleotide and one end of the polynucleotide comprising the round 1 barcode, thus attaching the barcode to the exposed polynucleotide. The proximity ligation may occur immediately following hybridization, in a subsequent step of the same cycle, or in a subsequent cycle (for example, while molecules comprising barcode 1B are ligated to polynucleotides in regions B). Thus, the one or more regions (e.g., regions A) on the substrate contain polynucleotides barcoded with the round 1 barcode (such as barcode 1A), while the one or more other regions (e.g., regions B) on the substrate do not contain polynucleotides barcoded with the round 1 barcode.

In some embodiments, the method comprises using a photoresist to generate a pattern. In some embodiments, the photoresist covering polynucleotides in the one or more other regions (e.g., regions B) is not removed prior to the next cycle where a photoresist is applied to cover polynucleotides in the one or more regions (e.g., regions A). In some embodiments, the photoresist covering polynucleotides in the one or more other regions (e.g., regions B) is removed prior to the next cycle. In some embodiments, the photoresist is removed from the substrate (e.g., all regions on the substrate) prior to the next cycle, and a new layer of a photoresist composition (which may be the same or different from the removed photoresist composition) is applied to the substrate, e.g., to cover both regions A and regions B.

In the next cycle, the polynucleotides barcoded with 1A in regions A are masked while regions B are unmasked to expose the polynucleotides in regions B. Another round 1 barcode (barcode 1B) may be attached to the exposed polynucleotides in regions B via hybridization and click chemistry based ligation, for example, as described above for attaching barcode 1A. In some embodiments, as shown in FIG. 2, a method disclosed herein comprises M rounds, where each round comprises N cycles (an exemplary cycle is shown in FIG. 4) to achieve a desired barcode diversity up to N^{M} wherein M and N are integers independent of each other and are at least 2.

In some embodiments, the barcode sequences received by the polynucleotides in feature(s) on the substrate in cycle I and in feature(s) on in cycle J are different, wherein I and J are integers and 1≤I≤J≤N. In some embodiments, the barcode sequences received by polynucleotides in feature(s) on the substrate in cycle I and in feature(s) on in cycle J are the same, wherein I and J are integers and 1≤I≤J≤N.

In some embodiments according to any one of the methods described herein, the irradiating and contacting steps are repeated in one or more cycles. In some embodiments, comprises a photoresist. In some embodiments, the photoresist is not removed prior to the one or more of the N cycles. In some embodiments, the photoresist is not removed during the one or more of the N cycles. In some embodiments, the photoresist is not removed between the one or more of the N cycles. In some embodiments, the method does not comprise re-applying a photoresist to the substrate prior to the one or more of the N cycles. In some embodiments, the method does not comprise re-applying a photoresist to the substrate during the one or more of the N cycles. In some embodiments, the method does not comprise re-applying a photoresist to the substrate during the one or more of the N cycles.

In some embodiments, the method comprises M rounds, wherein M is an integer of 2 or greater. In some embodiments, M is an integer of 4 or greater. In some embodiments, each of the M rounds comprises one or more cycles. In some embodiments, the method comprises removing changing the position of the photomask between each and/or after each round. In some embodiments, each of the M rounds comprises N cycles. In some embodiments, N≥3.

In some embodiments according to any one of the methods described herein, the polynucleotide molecules in a feature of the substrate receive a first barcode sequence in one of the cycles in round K, wherein K is an integer and I≤K<M. In some embodiments, the polynucleotide molecules in the feature comprising the first barcode sequence receive a second barcode sequence in one of the cycles in round (K+1), thereby forming polynucleotide molecules comprising the first and second barcode sequences. In some embodiments, the diversity of barcode sequences in the polynucleotides in a plurality of features on the substrate is N^{M}.

In some embodiments, the features on the substrate may correspond to regions of a substrate in which one or more barcodes have been incorporated. In some embodiments, the feature(s) may be no more than 0.5 micron, no more than 1 micron, no more than 5 microns, no more than 10 microns, or no more than 15 microns, no more than 20 microns, no more than 25 microns, no more than 30 microns, or no more than 35 microns, no more than 40 microns, no more than 45 microns, or no more than 50 microns in diameter. In some embodiments, the features on the substrate are below 10 microns in diameter (e.g., single cell scale resolution) and provide high throughput readout (e.g., by sequencing) for analyzing a sample, such as a tissue sample.

A method for providing an array, comprising: (a) contacting a lawn of polynucleotides immobilized on a substrate with a first barcode sequence, wherein the lawn of polynucleotides comprises a 3′-alkyne or 3′-azido group, and wherein the first barcode sequence comprises an polynucleotide with a 5′-azido or 5′-alkyne group that is a reaction partner for a Click reaction with the 3′-alkyne or 3′-azido group of the immobilized polynucleotides; (b) masking a first region of the immobilized polynucleotides on the substrate such that only a second region of the immobilized polynucleotides on the substrate are capable of being exposed to light; (c) irradiating the second region of immobilized polynucleotides on the substrate with a first ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the second region with the 5′-azido or 5′-alkyne groups of the first barcode sequence to generate a Click product comprising a 1,2,3-triazole group, thereby providing on the substrate an array comprising the first and second regions of the immobilized polynucleotides, wherein the second region of immobilized polynucleotides is barcoded with the first barcode sequence and the first region of immobilized polynucleotides is not barcoded with the first barcode.

In some embodiments, step (c) is conducted for less than about 10 minutes. In some embodiments, step (c) is conducted for about 2 minutes to about 10 minutes. In some embodiments, step (c) is conducted for about 2 minutes to about 5 minutes. In some embodiments, step (c) is steps conducted for less than 1, about 1, about 2, about 3, about 4, about 5, about 6, about 7, about 8, about 9, about 10, about 11, about 12, about 13, about 14, about 15 minutes.

In some embodiments, the contacting in step (a) is performed prior to the masking in step (b), as shown in FIG. 4. In some embodiments, the masking in step (b) is performed prior to the contacting in step (a), as shown in FIG. 1.

In any of the embodiments herein, the method further comprises contacting the lawn of polynucleotides immobilized on the substrate with a second barcode sequence, wherein the second barcode comprises an polynucleotide with a 5′-azido or 5′-alkyne group that is a reaction partner for a Click reaction with the 3′-alkyne or 3′-azido group of the immobilized polynucleotides. In some aspects, the second barcode sequence is different than the first barcode sequence. In any of the embodiments herein, the method further comprises masking a third region of the immobilized polynucleotides on the substrate such that only a fourth region of the immobilized polynucleotides on the substrate are capable of being exposed to light. In some aspects, the third region comprises some of the oligonucleotides in the first region. In some aspects, the third region comprises the Click products formed in step (c) of claim 1.

In any of the embodiments herein, the method further comprises irradiating the fourth region of immobilized polynucleotides on the substrate with a second ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the fourth region with the 5′-azido or 5′-alkyne groups of the second barcode sequence to generate a Click product comprising a 1,2,3-triazole group.

In any of the embodiments herein, the method further comprises repeating steps (a)-(c) for N cycles, wherein N is an integer of 2 or greater. In some aspects, all of the immobilized polynucleotides have been converted to Click products after N Cycles. In some aspects, the method comprises M rounds, M is an integer of 2 or greater, and each of the M rounds comprises one or more cycles. In some aspects, the sequence of the barcode in each cycle is different.

### V. Compositions, Kits, and Methods of Use

Also provided are compositions produced according to the methods described herein. These compositions include nucleic acid molecules and complexes, such as hybridization complexes, and kits and articles of manufacture (such as arrays) comprising such molecules and complexes.

In some embodiments, provided herein is an array comprising a lawn of polynucleotides immobilized on a substrate, wherein the polynucleotides in different regions of the substrate are covalently linked to different barcode sequences via a 1,2,3-triazole group. In some aspects, the 1,2,3-triazole linkages are formed from a Click reaction. In some aspects, the Click reaction is catalyzed by Cu (I).

In some embodiments, provided herein is a composition comprising: (i) a substrate comprising a first region and a second region, (ii) hybridization complexes in the first region, wherein at least one of the hybridization complexes comprise an polynucleotide molecule immobilized in the first region hybridized to a first splint, which is in turn hybridized to a first polynucleotide comprising a first barcode sequence, and (iii) polynucleotide molecules immobilized in the second region and protected by a from hybridization and ligation by a photomask.

In some embodiments, the composition comprises polynucleotides comprising 3′-alkyne or 3′-azido and/or 5′-azido or 5′-alkyne groups. The 3′-alkyne or 3′-azido and/or 5′-azido or 5′-alkyne groups are reaction partners for a Click reaction between two or more polynucleotides.

In some embodiments, the composition further comprises a copper compound (e.g., Cu (II) compound). In some embodiments, the copper compound is copper sulfate. In some aspects, irradiation in the presence of a Cu (II) compound and a photoinitiator generates Cu(I), which catalyzes the Click reaction between the 3′-alkyne or 3′-azido groups and the 5′-azido or 5′-alkyne groups of the polynucleotides. In some embodiments, the composition further comprises a Click product comprising a 1,2,3-triazole group.

In some embodiments, the composition further comprises a photoresist. In some embodiments, the photoresist forms a photoresist layer. In some embodiments, the polynucleotide molecules immobilized in the second region are embedded in the photoresist layer.

In some embodiments, provided herein is a composition, comprising a substrate comprising a plurality of universal polynucleotide molecules immobilized thereon, wherein the universal polynucleotide molecules in a first region of the substrate are available for hybridization and ligation, and the universal polynucleotide molecules in a second region of the substrate are protected from hybridization and ligation. In some embodiments, the composition further comprises a photomask masking the second region while exposing the first region to light. In some embodiments, the composition further comprises hybridization complexes in the first region, wherein at least one of the hybridization complexes comprise a universal polynucleotide molecule immobilized in the first region hybridized to a first splint, which is in turn hybridized to a first polynucleotide comprising a first barcode sequence.

Also provided herein are arrays comprising any one or more of the molecules, complexes, and/or compositions disclosed herein. Typically, an array includes at least two distinct nucleic acids that differ by monomeric sequence immobilized on, e.g., covalently to, different and known locations on the substrate surface. In certain embodiments, each distinct nucleic acid sequence of the array is typically present as a composition of multiple copies of the polymer on the substrate surface, e.g. as a spot on the surface of the substrate. The number of distinct nucleic acid sequences, and hence spots or similar structures, present on the array may vary, but is generally at least, usually at least 5 and more usually at least 10, where the number of different spots on the array may be as a high as 50, 100, 500, 1000, 10,000, 1,000,000, 10,000,000 or higher, depending on the intended use of the array. The spots of distinct polymers present on the array surface are generally present as a pattern, where the pattern may be in the form of organized rows and columns of spots, e.g. a grid of spots, across the substrate surface, a series of curvilinear rows across the substrate surface, e.g. a series of concentric circles or semi-circles of spots, and the like. The density of spots present on the array surface may vary, but is generally at least about 10 and usually at least about 100 spots/cm2, where the density may be as high as 106 or higher, or about 105 spots/cm2. In other embodiments, the polymeric sequences are not arranged in the form of distinct spots, but may be positioned on the surface such that there is substantially no space separating one polymer sequence/feature from another. The density of nucleic acids within an individual feature on the array may be as high as 1,000, 10,000, 25,000, 50,000, 100,000, 500,000, 1,000,000, or higher per square micron depending on the intended use of the array.

In some embodiments, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini, e.g. the 3′ or 5′ terminus.

Arrays can be used to measure large numbers of analytes simultaneously. In some embodiments, oligonucleotides are used, at least in part, to create an array. For example, one or more copies of a single species of oligonucleotide (e.g., capture probe) can correspond to or be directly or indirectly attached to a given feature in the array. In some embodiments, a given feature in the array includes two or more species of oligonucleotides (e.g., capture probes). In some embodiments, the two or more species of oligonucleotides (e.g., capture probes) attached directly or indirectly to a given feature on the array include a common (e.g., identical) spatial barcode.

In some embodiments, an array can include a capture probe attached directly or indirectly to the substrate. The capture probe can include a capture domain (e.g., a nucleotide sequence) that can specifically bind (e.g., hybridize) to a target analyte (e.g., mRNA, DNA, or protein) within a sample. In some embodiments, the binding of the capture probe to the target (e.g., hybridization) can be detected and quantified by detection of a visual signal, e.g., a fluorophore, a heavy metal (e.g., silver ion), or chemiluminescent label, which has been incorporated into the target. In some embodiments, the intensity of the visual signal correlates with the relative abundance of each analyte in the biological sample. Since an array can contain thousands or millions of capture probes (or more), an array can interrogate many analytes in parallel. In some embodiments, the binding (e.g., hybridization) of the capture probe to the target can be detected and quantified by creation of a molecule (e.g., cDNA from captured mRNA generated using reverse transcription) that is removed from the array, and sequenced.

Kits for use in analyte detection assays are provided. In some embodiments, the kit at least includes an array disclosed herein. The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the subject array assay devices for carrying out an array based assay. The instructions may be printed on a substrate, such as paper or plastic, etc. As such, the instructions may be present in the kits as a package insert, in the labeling of the container of the kit or components thereof (i.e., associated with the packaging or sub-packaging) etc.

The subject arrays find use in a variety of different applications, where such applications are generally analyte detection applications in which the presence of a particular analyte in a given sample is detected at least qualitatively, if not quantitatively. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc., and/or through sequencing of one or more components of the binding complex or a product thereof. The presence of the analyte in the sample is then deduced from the detection of binding complexes on the substrate surface, or sequence detection and/or analysis (e.g., by sequencing) on molecules indicative of the formation of the binding complex. In some embodiments, RNA molecules (e.g., mRNA) from a sample are captured by oligonucleotides (e.g., probes comprising a barcode and a poly(dT) sequence) on an array prepared by a method disclosed herein, cDNA molecules are generated via reverse transcription of the captured RNA molecules, and the cDNA molecules (e.g., a first strand cDNA) or portions or products (e.g., a second strand cDNA synthesized using a template switching oligonucleotide) thereof can be separated from the array and sequenced. Sequencing data obtained from molecules prepared on the array can be used to deduce the presence/absence or an amount of the RNA molecules in the sample.

Specific analyte detection applications of interest include hybridization assays in which the nucleic acid arrays of the subject invention are employed. In these assays, a sample of target nucleic acids or a tissue section is first prepared, where preparation may include labeling of the target nucleic acids with a label, e.g. a member of signal producing system. Following sample preparation, the sample is contacted with the array under hybridization conditions, whereby complexes are formed between target nucleic acids that are complementary to probe sequences attached to the array surface. The formation and/or presence of hybridized complexes is then detected, e.g., by analyzing molecules that are generated following the formation of the hybridized complexes, such as cDNA or a second strand generated from an RNA captured on the array. Specific hybridization assays of interest which may be practiced using the subject arrays include: gene discovery assays, differential gene expression analysis assays; nucleic acid sequencing assays, and the like.

### A. Spatial Analysis

In particular embodiments, provided herein are kits and compositions for spatial array-based analysis of biological samples. Array-based spatial analysis methods involve the transfer of one or more analytes or proxies thereof from a biological sample to an array of features on a substrate, where each feature is associated with a unique spatial location on the array. Subsequent analysis of the transferred analytes or proxies thereof includes determining the identity of the analytes and the spatial location of each analyte within the biological sample. The spatial location of each analyte within the biological sample is determined based on the feature to which each analyte or a proxy thereof is bound on the array, and the feature's relative spatial location within the array. In some embodiments, the array of features on a substrate comprise a spatial barcode that corresponds to the feature's relative spatial location within the array. Each spatial barcode of a feature may further comprise a fluorophore, to create a fluorescent hybridization array. A feature may comprise UMIs that are generally unique per nucleic acid molecule in the feature—this is so the number of unique molecules can be estimated, as opposed to an artifact in experiments or PCR amplification bias that drives amplification of smaller, specific nucleic acid sequences.

In some embodiments, an oligonucleotide probe can directly capture an analyte, such as mRNAs based on a poly(dT) capture domain on the oligonucleotide probe immobilized on an array. In some embodiments, the oligonucleotide probe is used for indirect analyte capture. For example, in fixed samples, such as FFPE, a probe pair can be used, and probes pairs can be target specific for each gene of the transcriptome. The probe pairs are delivered to a tissue section (which is itself on a spatial array) with a decrosslinking agent and a ligase, and the probe pairs are left to hybridize and ligate, thereby forming ligation products. The ligation products contain sequences in one or more overhangs of the probes, and the overhangs are not target specific and are complementary to capture domains on oligonucleotides immobilized on a spatial array, thus allowing the ligation product (which is a proxy for the analyte) to be captured on the array, processed, and subsequently analyzed (e.g., using a sequencing method).

In particular embodiments, the kits and compositions for spatial array-based analysis provide for the detection of differences in an analyte level (e.g., gene and/or protein expression) within different cells in a tissue of a mammal or within a single cell from a mammal. For example, the kits and compositions can be used to detect the differences in analyte levels (e.g., gene and/or protein expression) within different cells in histological slide samples (e.g., intact tissue section), the data from which can be reassembled to generate a three-dimensional map of analyte levels (e.g., gene and/or protein expression) of a tissue sample obtained from a mammal, e.g., with a degree of spatial resolution (e.g., single-cell scale resolution).

In some embodiments, an array generated using a method disclosed herein can be used in array-based spatial analysis methods which involve the transfer of one or more analytes from a biological sample to an array of features on a substrate, each of which is associated with a unique spatial location on the array. Subsequent analysis of the transferred analytes includes determining the identity of the analytes and the spatial location of each analyte within the sample. The spatial location of each analyte within the sample is determined based on the feature to which each analyte is bound in the array, and the feature's relative spatial location within the array.

There are at least two general methods to associate a spatial barcode with one or more neighboring cells, such that the spatial barcode identifies the one or more cells, and/or contents of the one or more cells, as associated with a particular spatial location. One general method is to drive target analytes out of a cell and towards the spatially-barcoded array. In some embodiments, the spatially-barcoded array populated with capture probes is contacted with a sample, and sample is permeabilized, allowing the target analyte to migrate away from the sample and toward the array. The target analyte interacts with a capture probe on the spatially-barcoded array. Once the target analyte hybridizes/is bound to the capture probe, the sample is optionally removed from the array and the capture probes are analyzed in order to obtain spatially-resolved analyte information. Methods for performing such spatial analysis of tissue sections include but are not limited to those methods disclosed in U.S. Pat. Nos. 10,030,261, 11,332,790 and US Patent Pub No. 20220127672 and US Patent Pub No. 20220106632, the contents of which are herein incorporated by reference in their entireties.

Another general method is to cleave the spatially-barcoded capture probes from an array, and drive the spatially-barcoded capture probes towards and/or into or onto the sample. In some embodiments, the spatially-barcoded array populated with capture probes is contacted with a sample. The spatially-barcoded capture probes are cleaved and then interact with cells within the provided sample (See, for example, U.S. Pat. No. 11,352,659 the contents of which are herein incorporate by reference in its entirety). The interaction can be a covalent or non-covalent cell-surface interaction. The interaction can be an intracellular interaction facilitated by a delivery system or a cell penetration peptide. Once the spatially-barcoded capture probe is associated with a particular cell, the sample can be optionally removed for analysis. The sample can be optionally dissociated before analysis. Once the tagged cell is associated with the spatially-barcoded capture probe, the capture probes can be analyzed (e.g., by sequencing) to obtain spatially-resolved information about the tagged cell.

Sample preparation may include placing the sample on a slide, fixing the sample, and/or staining the sample for imaging. The stained sample may be imaged on the array using both brightfield (to image the sample hematoxylin and eosin stain) and/or fluorescence (to image features) modalities. In some embodiments, target analytes are then released from the sample and capture probes forming the spatially-barcoded array hybridize or bind the released target analytes. The sample is then removed from the array and the capture probes cleaved from the array. The sample and array are then optionally imaged a second time in one or both modalities (brightfield and fluorescence) while the analytes are reverse transcribed into cDNA, and an amplicon library is prepared and sequenced. The two sets of images can then be spatially-overlaid in order to correlate spatially-identified sample information. When the sample and array are not imaged a second time, a spot coordinate file may be supplied. The spot coordinate file can replace the second imaging step. Further, amplicon library preparation can be performed with a unique PCR adapter and sequenced.

In some embodiments, a spatially-labelled array on a substrate is used, where capture probes labelled with spatial barcodes are clustered at areas called features. The spatially-labelled capture probes can include a cleavage domain, one or more functional sequences, a spatial barcode, a unique molecular identifier, and a capture domain. The spatially-labelled capture probes can also include a 5′ end modification for reversible attachment to the substrate. The spatially-barcoded array is contacted with a sample, and the sample is permeabilized through application of permeabilization reagents. Permeabilization reagents may be administered by placing the array/sample assembly within a bulk solution. Alternatively, permeabilization reagents may be administered to the sample via a diffusion-resistant medium and/or a physical barrier such as a lid, wherein the sample is sandwiched between the diffusion-resistant medium and/or barrier and the array-containing substrate. The analytes are migrated toward the spatially-barcoded capture array using any number of techniques disclosed herein. For example, analyte migration can occur using a diffusion-resistant medium lid and passive migration. As another example, analyte migration can be active migration, using an electrophoretic transfer system, for example. Once the analytes are in close proximity to the spatially-barcoded capture probes, the capture probes can hybridize or otherwise bind a target analyte. The sample can be optionally removed from the array.

Adapters and assay primers can be used to allow the capture probe or the analyte capture agent to be attached to any suitable assay primers and used in any suitable assays. A capture probe that includes a spatial barcode can be attached to a bead that includes a poly(dT) sequence. A capture probe including a spatial barcode and a poly(T) sequence can be used to assay multiple biological analytes as generally described herein (e.g., the biological analyte includes a poly(A) sequence or is coupled to or otherwise is associated with an analyte capture agent comprising a poly(A) sequence as the analyte capture sequence).

The capture probes can be optionally cleaved from the array, and the captured analytes can be spatially-tagged by performing a reverse transcriptase first strand cDNA reaction. A first strand cDNA reaction can be optionally performed using template switching oligonucleotides. For example, a template switching oligonucleotide can hybridize to a poly(C) tail added to a 3′end of the cDNA by a reverse transcriptase enzyme. The original mRNA template and template switching oligonucleotide can then be denatured from the cDNA and the barcoded capture probe can then hybridize with the cDNA and a complement of the cDNA can be generated. The first strand cDNA can then be purified and collected for downstream amplification steps. The first strand cDNA can be amplified using PCR, wherein the forward and reverse primers flank the spatial barcode and target analyte regions of interest, generating a library associated with a particular spatial barcode. In some embodiments, the cDNA comprises a sequencing by synthesis (SBS) primer sequence. The library amplicons are sequenced and analyzed to decode spatial information.

In some embodiments, the sample is removed from the spatially-barcoded array and the spatially-barcoded capture probes are removed from the array for barcoded analyte amplification and library preparation. Another embodiment includes performing first strand synthesis using template switching oligonucleotides on the spatially-barcoded array without cleaving the capture probes. Once the capture probes capture the target analyte(s), first strand cDNA created by template switching and reverse transcriptase is then denatured and the second strand is then extended. The second strand cDNA is then denatured from the first strand cDNA, neutralized, and transferred to a tube. cDNA quantification and amplification can be performed using standard techniques discussed herein. The cDNA can then be subjected to library preparation and indexing, including fragmentation, end-repair, and a-tailing, and indexing PCR steps, and then sequenced.

### VI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used in this specification and the appended claims, the singular forms “a,” “an” and “the” include plural referents unless the context clearly dictates otherwise. Thus, for example, reference to “a molecule” includes a plurality of such molecules, and the like.

The term “barcode,” comprises a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell scale resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

As used herein, the term “substrate” generally refers to a substance, structure, surface, material, means, or composition, which comprises a nonbiological, synthetic, nonliving, planar, spherical or flat surface. The substrate may include, for example and without limitation, semiconductors, synthetic metals, synthetic semiconductors, insulators and dopants; metals, alloys, elements, compounds and minerals; synthetic, cleaved, etched, lithographed, printed, machined and microfabricated slides, devices, structures and surfaces; industrial polymers, plastics, membranes; silicon, silicates, glass, metals and ceramics; wood, paper, cardboard, cotton, wool, cloth, woven and nonwoven fibers, materials and fabrics; nanostructures and microstructures. The substrate may comprises an immobilization matrix such as but not limited to, insolubilized substance, solid phase, surface, layer, coating, woven or nonwoven fiber, matrix, crystal, membrane, insoluble polymer, plastic, glass, biological or biocompatible or bioerodible or biodegradable polymer or matrix, microparticle or nanoparticle. Other example may include, for example and without limitation, monolayers, bilayers, commercial membranes, resins, matrices, fibers, separation media, chromatography supports, polymers, plastics, glass, mica, gold, beads, microspheres, nanospheres, silicon, gallium arsenide, organic and inorganic metals, semiconductors, insulators, microstructures and nanostructures. Microstructures and nanostructures may include, without limitation, microminiaturized, nanometer-scale and supramolecular probes, tips, bars, pegs, plugs, rods, sleeves, wires, filaments, and tubes.

As used herein, the term “nucleic acid” generally refers to a polymer comprising one or more nucleic acid subunits or nucleotides. A nucleic acid may include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double-stranded.

The term “nucleic acid sequence” or “nucleotide sequence” as used herein generally refers to nucleic acid molecules with a given sequence of nucleotides, of which it may be desired to know the presence or amount. The nucleotide sequence can comprise ribonucleic acid (RNA) or DNA, or a sequence derived from RNA or DNA. Examples of nucleotide sequences are sequences corresponding to natural or synthetic RNA or DNA including genomic DNA and messenger RNA. The length of the sequence can be any length that can be amplified into nucleic acid amplification products, or amplicons, for example, up to about 20, 50, 100, 200, 300, 400, 500, 600, 700, 800, 1000, 1200, 1500, 2000, 5000, 10000 or more than 10000 nucleotides in length, or at least about 20, 50, 100, 200, 300, 400, 500, 600, 700, 800, 1000, 1200, 1500, 2000, 5000, 10000 nucleotides in length.

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (i.e., can be oligoribonucleotides) and/or deoxyribonucleotide monomers (i.e., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

As used herein, the term “adjacent” or “adjacent to,” includes “next to,” “adjoining,” and “abutting.” In one example, a first location is adjacent to a second location when the first location is in direct contact and shares a common border with the second location and there is no space between the two locations. In some cases, the adjacent is not diagonally adjacent.

An “adaptor,” an “adapter,” and a “tag” are terms that are used interchangeably in this disclosure, and refer to species that can be coupled to a polynucleotide sequence (in a process referred to as “tagging”) using any one of many different techniques including (but not limited to) ligation, hybridization, and tagmentation. Adaptors can also be nucleic acid sequences that add a function, e.g., spacer sequences, primer sequences/sites, barcode sequences, unique molecular identifier sequences.

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

A “proximity ligation” is a method of ligating two (or more) nucleic acid sequences that are in proximity with each other through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference).

A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

As used herein, the term “splint” is an oligonucleotide that, when hybridized to other polynucleotides, acts as a “splint” to position the polynucleotides next to one another so that they can be ligated together. In some embodiments, the splint is DNA or RNA. The splint can include a nucleotide sequence that is partially complimentary to nucleotide sequences from two or more different oligonucleotides. In some embodiments, the splint assists in ligating a “donor” oligonucleotide and an “acceptor” oligonucleotide. In general, an RNA ligase, a DNA ligase, or another other variety of ligase is used to ligate two nucleotide sequences together

In some embodiments, the splint is between 10 and 50 oligonucleotides in length, e.g., between 10 and 45, 10 and 40, 10 and 35, 10 and 30, 10 and 25, or 10 and 20 oligonucleotides in length. In some embodiments, the splint is between 15 and 50, 15 and 45, 15 and 40, 15 and 35, 15 and 30, 15 and 30, or 15 and 25 nucleotides in length.

A “feature” is an entity that acts as a support or repository for various molecular entities used in sample analysis. In some embodiments, some or all of the features in an array are functionalized for analyte capture. In some embodiments, functionalized features include one or more capture probe(s). Examples of features include, but are not limited to, a bead, a spot of any two- or three-dimensional geometry (e.g., an ink jet spot, a masked spot, a square on a grid), a well, and a hydrogel pad. In some embodiments, features are directly or indirectly attached or fixed to a substrate. In some embodiments, the features are not directly or indirectly attached or fixed to a substrate, but instead, for example, are disposed within an enclosed or partially enclosed three dimensional space (e.g., wells or divots).

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “template” as used herein generally refers to individual polynucleotide molecules from which another nucleic acid, including a complementary nucleic acid strand, can be synthesized by a nucleic acid polymerase. In addition, the template can be one or both strands of the polynucleotides that are capable of acting as templates for template-dependent nucleic acid polymerization catalyzed by the nucleic acid polymerase. Use of this term should not be taken as limiting the scope of the present disclosure to polynucleotides which are actually used as templates in a subsequent enzyme-catalyzed polymerization reaction. The template can be an RNA or DNA. The template can be cDNA corresponding to an RNA sequence. The template can be DNA.

As used herein, “amplification” of a template nucleic acid generally refers to a process of creating (e.g., in vitro) nucleic acid strands that are identical or complementary to at least a portion of a template nucleic acid sequence, or a universal or tag sequence that serves as a surrogate for the template nucleic acid sequence, all of which are only made if the template nucleic acid is present in a sample. Typically, nucleic acid amplification uses one or more nucleic acid polymerase and/or transcriptase enzymes to produce multiple copies of a template nucleic acid or fragments thereof, or of a sequence complementary to the template nucleic acid or fragments thereof. In vitro nucleic acid amplification techniques are may include transcription-associated amplification methods, such as Transcription-Mediated Amplification (TMA) or Nucleic Acid Sequence-Based Amplification (NASBA), and other methods such as Polymerase Chain Reaction (PCR), Reverse Transcriptase-PCR (RT-PCR), Replicase Mediated Amplification, and Ligase Chain Reaction (LCR).

In addition to those above, a wide variety of other features can be used to form the arrays described herein. For example, in some embodiments, features that are formed from polymers and/or biopolymers that are jet printed, screen printed, or electrostatically deposited on a substrate can be used to form arrays.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for providing an array, comprising:
(a) contacting a lawn of polynucleotides immobilized on a substrate with a first barcode sequence, wherein the polynucleotides comprise a 3′-alkyne or 3′-azido group, and wherein the first barcode sequence comprises an oligonucleotide with a 5′-azido or 5′-alkyne group that is a reaction partner for a Click reaction with the 3′-alkyne or 3′-azido group of the immobilized polynucleotides;
(b) masking a first region of the lawn of polynucleotides on the substrate such that only a second region of the lawn of polynucleotides on the substrate are capable of being exposed to light;
(c) irradiating the second region with a first ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the second region with the 5′-azido or 5′-alkyne groups of the first barcode sequence to generate a Click product comprising a 1,2,3-triazole group, wherein molecules of the first barcode sequence in proximity to the immobilized polynucleotides in the first region are not catalyzed by the Cu(I) to undergo the Click reaction,
thereby providing on the substrate an array comprising the first and second regions of the lawn of polynucleotides, wherein the immobilized polynucleotides in the second region is barcoded with the first barcode sequence and the immobilized polynucleotides in the first region is not barcoded with the first barcode sequence.

2. The method of claim 1, wherein the immobilized polynucleotides comprise a 3′-alkyne group.

3. The method of claim 2, wherein the first barcode sequence comprises a 5′-azido group.

4. The method of claim 1, wherein the immobilized polynucleotides comprise a 3′-azido group.

5. The method of claim 4, wherein the first barcode sequence comprises a 5′-alkyne group.

6. The method of claim 1, wherein said contacting in step (a) comprises flowing the first barcode sequence over the lawn of immobilized polynucleotides.

7. The method of claim 1, further comprising contacting the lawn of immobilized polynucleotides with a splint, wherein the splint comprises a first nucleotide sequence that is complementary to a nucleotide sequence on the immobilized polynucleotides.

8. The method of claim 7, wherein the splint comprises a second nucleotide sequence that is complementary to a nucleotide sequence on the first barcode sequence.

9. The method of claim 7, wherein the immobilized polynucleotides are ligated to the first barcode sequence using the splint as template.

10. The method of claim 9, further comprising removing i) the splint and ii) molecules of the first barcode sequence that are brought into proximity to the immobilized polynucleotides in the first region but are not ligated to the immobilized polynucleotides in the first region, after the ligation of molecules of the first barcode sequence to the immobilized polynucleotides in the second region.

11. The method of claim 7, wherein contacting the splint with the lawn of immobilized comprises flowing the splint over the lawn of immobilized polynucleotides.

12. The method of claim 1, wherein the Cu (II) compound is copper sulfate.

13. The method of claim 1, further comprising contacting the lawn of polynucleotides with a free radical scavenger.

14. The method of claim 1, wherein the Cu (II) compound and the photoinitiator are flowed over the immobilized lawn of polynucleotides in a solution.

15. The method of claim 14, wherein the solution comprises DMSO.

16-20. (canceled)

21. The method of claim 1, wherein the irradiation in step (b) comprises using a photomask to selectively irradiate the second region of the immobilized polynucleotides.

22. The method of claim 1, further comprising capping the unreacted 3′ groups in the second region of the immobilized polynucleotides.

23-26. (canceled)

27. The method of claim 1, further comprising contacting the lawn of polynucleotides immobilized on the substrate with a second barcode sequence, wherein the second barcode sequence comprises an oligonucleotide with a 5′-azido or 5′-alkyne group that is a reaction partner for a Click reaction with the 3′-alkyne or 3′-azido group of the immobilized polynucleotides.

28. The method of claim 27, wherein the second barcode sequence is different than the first barcode sequence.

29-32. (canceled)

33. The method of claim 27, further comprising repeating steps (a)-(c) for N cycles, wherein N is an integer of 2 or greater.

34. The method of claim 33, wherein the sequence of the barcode sequence in each cycle is different.

35. The method of claim 33, wherein the method comprises M rounds, M is an integer of 4 or greater, and each of the M rounds comprises one or more cycles.

36. (canceled)

37. A method for providing an array, comprising:
(a) irradiating a substrate comprising an unmasked first region and a masked second region, whereby a photoresist in the first region is degraded to render immobilized polynucleotide molecules in the first region available for hybridization and/or ligation, whereas immobilized polynucleotide molecules in the second region are protected by a photoresist from hybridization and/or ligation, wherein polynucleotide molecules in the first and second regions comprise a 3′-alkyne or 3′-azido group;
(b) contacting the immobilized polynucleotide molecules in the first region with a first barcode sequence, wherein the first barcode sequence comprises an oligonucleotide with a 5′-azido or 5′-alkyne group that is a reaction partner for a Click reaction with the 3′-alkyne or 3′-azido group of the immobilized polynucleotides in the first region, and wherein polynucleotide molecules in the second region do not receive the barcode sequence;
(c) irradiating the first region of the substrate with a first ultraviolet light in the presence of a Cu (II) compound and a photoinitiator, thereby generating Cu(I), wherein the Cu(I) catalyzes the Click reaction between the 3′-alkyne or 3′-azido groups of the immobilized polynucleotides in the first region of the substrate with the 5′-azido or 5′-alkyne groups of the first barcode sequence to generate a Click product comprising a 1,2,3-triazole group,
thereby providing on the substrate an array comprising different polynucleotide molecules in the first and second regions.

38-77. (canceled)
